(19) United States
(12) Patent Application Publication
(10) Pub. No.: US 2007/0270578 A1
(43) Pub. Date: Nov. 22, 2007

(54) Plants Having Increased Yield and a Method for Making the Same

(75) Inventor: Valerie Frankard, Waterloo (BE)

Correspondence Address: CONNOLLY BOVE LODGE & HUTZ, LLP, P O BOX 2207, WILMINGTON, DE, 19899, US

(73) Assignee: CropDesign N.V., Zwijnaarde (BE)

(21) Appl. No.: 11/791,499

(22) PCT Filed: Nov. 24, 2005
(86) PCT No.: PCT/EP05/56202

§ 371 Date: Jun. 21, 2007

Related U.S. Application Data
(60) Provisional application No. 60/632,273, filed on Dec. 1, 2004.

(30) Foreign Application Priority Data
Nov. 25, 2004 (EP) 04106065.8

(51) Int. Cl. A01H 5/00; C12N 15/82

(52) U.S. Cl. 536/22.1; 800/290; 800/298; 800/320; 800/320.1; 800/320.2; 800/320.3; 435/468

(57) ABSTRACT

The present invention concerns a method for increasing plant yield by increasing activity in a plant of an Os-MADS18-like polypeptide or a homologue thereof. One such method comprises introducing into a plant an OsMADS18-like nucleic acid or variant thereof. The invention also relates to transgenic plants having introduced therein an OsMADS18-like nucleic acid or variant thereof, which plants have increased yield relative to corresponding wild type plants. The present invention also concerns constructs useful in the methods of the invention.

The present invention relates generally to the field of molecular biology and concerns a method for increasing plant yield relative to corresponding wild type plants. More specifically, the present invention concerns a method for increasing plant yield by increasing activity in a plant of an Oryza sativa (Os)MADS18-like polypeptide or a homologue thereof. The present invention also concerns plants having increased activity of an OsMADS18-like polypeptide or a homologue thereof, which plants have increased yield relative to corresponding wild type plants. The invention also provides constructs useful in the methods of the invention.

The ever-increasing world population and the dwindling supply of arable land available for agriculture fuel research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Optimizing one of the abovementioned factors may therefore increase crop yield.

The ability to increase plant yield would have many applications in areas such as agriculture, including in the production of ornamental plants, arboriculture, horticulture and forestry.

It has now been found that increasing activity in a plant of an OsMADS18-like polypeptide gives plants having increased yield relative to corresponding wild type plants.

The MADS-box genes (MCM1 in yeast, Agamous and Deficiens in plants, and SRF (serum response factor) in humans) constitute a large gene family of eukaryotic transcriptional regulators involved in diverse aspects of yeast, plant and animal development. MADS-box genes encode a strongly conserved MADS domain responsible for DNA binding to specific boxes in the regulatory region of their target genes. The gene family can be divided into two main lineages, type I and type II. Type II genes are also named MIKC-type proteins, referring to the four functional domains they possess (see FIG. 1, from Jack (2001) Plant Molec Biol 46: 515-520):
MADS for DNA binding, about 60 amino acids (highly conserved) located at the N-terminal end of the protein;
I for intervening domain (less conserved), involved in the selective formation of MADS dimer;
K for keratin domain (well conserved) responsible for dimerization;
C for C-terminal region (variable in sequence and length) involved in transcriptional activation, or in the formation of a multimeric transcription factor complex.

Over 100 MADS-box genes have been identified in Arabidopsis, and have been phylogenetically classified into 12 clades, each dade having specific deviations from the MADS consensus (Thiessen et al. (1996) J Mol Evol 43: 484-516). OsMADS18 (formerly called FDRMADS7 or OsMADS28) belongs to the SQUA dade (for SQUAMOSA, from Antirrhinum majus), along with the following Arabidopsis genes: FUL/Ag18 (FRUITFULL), CAL/Agl10 (CAULIFLOWER), AP1/Agl7 (APETALA1) and the less characterized MADS AGL79. Genes of the SQUA dade are A function organ identity genes with reference to the ABC floral organ identity specification model as proposed by Coen and Meyerowitz in 1991 (Nature 353: 31-7). Rice possesses four A-group genes: OsMADS14, OsMADS15, OsMADS18, and OsMADS20, each being member of the SQUA lade.

The SQUA dade in dicots is subdivided into two subgroups, the AP1 and the FUL subclades. These subclades diverge essentially with respect to the specific amino acid motifs located at the C-terminus of their respective proteins (Litt and Irish (2003) Genetics 165:821-833). In addition to the presence of a specific AP1 amino acid motif, the dicot AP1 dade related proteins usually comprise a farnesylation motif at their C-terminus (this motif is CAAX, where C is cysteine, A is usually an aliphatic amino acid, and X is methionine, glutamine, serine, cysteine or alanine). In monocots, the SQUA dade proteins are also subdivided into two main groups, which may be distinguished based on conserved C-terminal motifs located within the last 15 amino acids of the proteins: LPPWMLRT (SEQ ID NO: 18) and LPPWMLSH (SEQ ID NO: 19) (FIG. 2). In contrast to dicot sequences of the SQUA clade, monocot sequences of the SQUA dade do not possess a farnesylation motif at their C-terminus.

OsMADS18 clusters up with the corn ZMM28 polypeptide and the barley m3 polypeptide (Becker and Thiessen (2003) Mol Phylogenet Evol 29(3): 464-89). All three proteins comprise the LPPWMLRT amino acid motif within about the last 15 amino acids of their C-terminus. Two other rice MADS box proteins from the SQUA clade, OsMADS14 and OsMADS15, belong to the subclade with the LPPWMLSH motif within about the last 15 amino acids of their C-terminus. For both of these motifs, LPPWMLRT (SEQ ID NO: 18) and LPPWMLSH (SEQ ID NO: 19), the most conserved amino acids are located in the second (P for proline) and fourth (W for tryptophan) positions.

OsMADS18 is a widely expressed gene and its RNA can be detected in root, leaf, inflorescence and developing kernel tissue (Masiero et aL (2002) J Biol Chem 277(29): 26429-35). OsMADS18 may be involved in the transition from vegetative growth to flowering (Fornara et al. (2004) Plant Physiol 135(4): 2207-19). Constitutive overexpression of OsMADS18 in rice leads to dwarfed plants and earlier flowering as a consequence of accelerated plant maturation.

In yeast two hybrid experiments, OsMADS18 has been shown to form heterodimers with OsMADS6, OsMADS24, OsMADS45 and OsMADS47. It also specifically interacts with OsNF-YB1, which shares the highest sequence identity with Arabidopsis Leafy Cotyledon1 (LEC1 or AtNF-YB9). Ternary complex formation between the three proteins OsMADS18, OsMADS6 and OsNF-YB1 has also been shown (Masiero et al. (2002) J Biol Chem 277(29): 26429-35).

In U.S. Pat. No. 6,229,068 B1, Yanofsky et al. describe a method to increase seed (or fruit) size in a plant by using an AGL8-related gene product and ectopically expressing it in the plant. Preferred regulatory elements mentioned in the document for the ectopic expression of an AGL8-related gene product are constitutive, seed-preferred or inducible elements.

International patent application WO 02/33091 discloses a monocot (from perennial ryegrass) MADS-box transcription factor for the manipulation of flowering and plant architecture. International patent application WO 03/057877 discloses a MADS-box cDNA having a single nucleotide polymorphism (SNP) among different barley varieties.

According to one embodiment of the present invention, there is provided a method for increasing plant yield, comprising increasing activity in a plant of an OsMADS18-like polypeptide or a homologue thereof, which OsMADS18-like polypeptide or homologue thereof is: (i) a monocot type 11 MADS-box transcription factor of the SQUAMOSA clade; and (ii) has DNA and protein binding activity; and (iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and (iv) has at least 65% sequence identity to the OsMADS18 protein of SEQ ID NO: 2.

Advantageously, performance of the methods according to the present invention results in plants having increased yield, particularly seed yield.

The term “increased yield” as defined herein is taken to mean an increase in any one or more of the following, each relative to corresponding wild type plants: (i) increased biomass (weight) of one or more parts of a plant, particularly aboveground (harvestable) parts or increased biomass of any other harvestable part; (ii) increased seed yield, which includes an increase in seed biomass (seed weight) and which may be an increase in the seed weight per plant or on an individual seed basis or an increase in seed weight per hectare or acre; (iii) increased number of flowers (florets) per panicle, which is expressed as a ratio of the number of filled seeds over the number of primary panicles; (iv) increased number of (filled) seeds; (v) increased fill rate of seeds (which is the number of filled seeds divided by the total number of seeds and multiplied by 100); (vi) increased seed size, which may also influence the composition of seeds; (vii) increased seed volume, which may also influence the composition of seeds (for example due to an increase in amount or a change in the composition of oil, protein or carbohydrate); (viii) increased seed area; (ix) increased seed length; (x) increased seed width; (x) increased seed perimeter; (xi) increased harvest index, which is expressed as a ratio of the yield of harvestable parts, such as seeds, over the total biomass; and (xii) increased thousand kernel weight (TKW), which is extrapolated from the number of filled seeds counted and their total weight. An increased TKW may result from an increased seed size and/or seed weight and may also result from an increase in embryo size and/or endosperm size.

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in thousand kernel weight, among others. An increase in yield may also result in modified architecture, or may occur as a result of modified architecture.

According to a preferred feature, performance of the methods of the invention result in plants having increased seed yield. Therefore, according to the present invention, there is provided a method for increasing plant seed yield, which method comprises increasing activity in a plant of an OsMADS18-like polypeptide or a homologue thereof.

Since the transgenic plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts of a plant (including seeds), or may be throughout substantially the whole plant. A plant having an increased growth rate may even exhibit early flowering. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soy bean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing yield in plants, which method comprises increasing activity in a plant of an OsMADS18-like polypeptide or a homologue thereof.

An increase in yield and/or growth rate occurs whether the plant is under non-stress conditions or whether the plant is exposed to various stresses compared to control/wild type plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be everyday biotic and/or abiotic (environmental) stresses. Typical abiotic or environmental stresses include temperature stresses caused by atypical hot or cold/freezing temperatures; salt stress; water stress (drought or excess water). Chemicals may also cause abiotic stresses. Biotic stresses are typically those stresses caused by pathogens, such as bacteria, viruses, fungi and insects.

The abovementioned increases in yield may advantageously be obtained in any plant upon performance of the methods of the invention.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen, and microspores, again wherein each of the aforementioned comprise the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plunjuga, Betula spp., Brassica spp., Bruguiera gymnorrhiza, Burkea africana, Butea frondosa, Cadaba fannosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divancata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimper, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squaffosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugarcane, sunflower, tomato, squash, tea and algae, amongst others. According to a preferred embodiment of the present invention, the plant is a crop plant such as soybean, sunflower, canola, alfalfa, rapeseed, cotton, tomato, potato or tobacco. Further preferably, the plant is a monocotyledonous plant, such as sugar cane. More preferably the plant is a cereal, such as rice, maize, wheat, barley, millet, rye, sorghum or oats.

The activity of an OsMADS18-like polypeptide may be increased by raising levels of the polypeptide. OsMADS18 mRNA levels may also be increased. Alternatively, activity may also be increased when there is no change in levels of an OsMADS18-like polypeptide, or even when there is a reduction in levels of an OsMADS18-like polypeptide. This may occur when the intrinsic properties of the polypeptide are altered, for example, by making mutant versions that are more active that the wild type polypeptide.

The term “OsMADS18-like polypeptide or a homologue thereof” as defined herein refers to a polypeptide (i) being a monocot type II MADS-box transcription factor of the SQUAMOSA lade; and (ii) having DNA and protein binding activity; and (iii) comprising the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and (iv) having at least 65% sequence identity to the OsMADS18 protein of SEQ ID NO: 2. The substitution in the motif may be a conservative substitution but is not limited to conservative substitutions. The conservative amino acid substitution may replace any one or more of the amino acid residues of the aforementioned motif, including at positions 2 and 4 as occupied by proline and tryptophan respectively. Conservative substitution tables are readily available in the art. The table below gives examples of conserved amino acid substitutions.

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

An “OsMADS18-like polypeptide or a homologue thereof” may readily be identified using routine techniques well known in the art. For example, DNA-binding activity and protein-protein interactions may readily be determined in vitro or in vivo using techniques well known in the art. Examples of in vitro assays for DNA binding activity include: gel retardation analysis using known MADS-box DNA binding domains (West et al. (1998) NucI Acid Res 26(23): 5277-87), or yeast one-hybrid assays. An example of an in vitro assay for protein-protein interactions is the yeast two-hybrid analysis (Fields and Song (1989) Nature 340:245-6).

Furthermore, the motif defined above (SEQ ID NO: 18) may also be readily identified by a person skilled in the art simply by making an alignment and searching for the motif at the C-terminal end of a polypeptide. Similarly, a polypeptide having at least 65% identity to the amino acid sequence represented by SEQ ID NO: 2 may also readily be established by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information. Homologues of OsMADS18 comprising the motif LPPWMLRT (SEQ ID NO: 18) within the last 15 amino acids of the protein, and having at least 65% identity to the amino acid sequences represented by SEQ ID NO: 2 may readily be identified using, for example, the ClustalW multiple sequence alignment algorithm (version 1.83) available at http://clustalw.genomejp/sit-bin/nph-clustalw, with the following pairwise alignment parameters: K-tuple (word) size of 1, window size of 5, gap penalty of 4, number of top diagonal of 5, and a scoring method in percentage.

Examples of plant-derived polypeptides covered by the term “OsMADS18-like polypeptide or a homologue thereof” include (see also Table 2): OsMADS18 AF091458 from Oryza sativa (SEQ ID NO: 2), ZMM28 (or m28) AJ430695 from Zea mays (SEQ ID NO: 4), MADS3 AY198328 from Lolium perenne (SEQ ID NO: 6), m3 AJ249143 from Hordeum vulgare (SEQ ID NO: 8). A ZMM28-like protein was deduced from a contig of several Saccharum officinarum overlapping ESTs, CA285442 (SEQ ID NO: 9), CA200888 (SEQ ID NO: 10), CA247266 (SEQ ID NO: 11), CA282968 (SEQ ID NO: 12), and CA299090 (SEQ ID NO: 13). The Saccharum officinarum protein (SEQ ID NO: 15) was deduced from the consensus sequence (SEQ ID NO: 14) obtained by aligning five different ESTs. SEQ ID NO: 16 is a second nucleotide consensus sequence with one nucleotide change (G to T) at position 578 bp from the ATG, compared to SEQ ID NO: 14. The polypeptide SEQ ID NO: 17 was deduced from SEQ ID NO: 16, and is identical to SEQ ID NO: 15 except for one amino acid change at position 193 of the protein (V193G).

TABLE 2
Examples of OsMADS18-like monocot orthologues

| | | Accession | DNA | Protein | |
|---|---|---|---|---|---|
| | Gene name | number | SEQ ID N° | SEQ ID NO | Source |
| 1 | OsMADS18 | AF091458 | 1 | 2 | Oryza sativa |
| 2 | ZMM28 | AJ430695 | 3 | 4 | Zea mays |
| 3 | MADS3 | AY198328 | 5 | 6 | Lolium perenne |
| 4 | m3 | AJ249143 | 7 | 8 | Hordeum vulgare |
| 5 | ZMM28-like | CA285442 | 14, 16 | 15, 17 | Saccharum officinarum |
| | | CA200888 | (predicted) | (predicted) | |
| | | CA247266 | | | |
| | | CA282968 | | | |
| | | CA299090 | | | |

It is to be understood that sequences falling under the definition of “OsMADS18-like polypeptide or homologue thereof” are not to be limited to the sequences represented by SEQ ID NO: 2, SEQ ID NO: 4, SEQ ID NO: 6, SEQ ID NO: 8, SEQ ID NO: 15 or SEQ ID NO: 17, but that any polypeptide meeting the criteria of: (i) being a monocot type 11 MADS-box transcription factor of the SQUAMOSA clade; and (ii) having DNA and protein binding activity; and (iii) comprising the motif LPPWMLRT (SEQ ID NO: 18) in the last 15 amino acids at the C terminus of the protein, allowing for one amino acid substitution anywhere in the motif but not in the second and fourth positions of the motif, which are respectively a proline and a tryptophan; and (iv) having at least 65% sequence identity to the OsMADS18 protein of SEQ ID NO: 2 may be suitable for use in the methods of the invention and to obtain plants having increased yield relative to corresponding wild type plants.

The nucleic acid encoding an OsMADS18-like polypeptide or a homologue thereof may be any natural or synthetic nucleic acid. Therefore the term “OsMADS18-like nucleic acid/gene” as defined herein is any nucleic acid/gene encoding an OsMADS18-like polypeptide or a homologue thereof as defined hereinabove. Examples of OsMADS18-like nucleic acids include those represented by any one of SEQ ID NO: 1, SEQ ID NO: 3, SEQ ID NO: 5, SEQ ID NO: 7, SEQ ID NO: 14 and SEQ ID NO: 16. OsMADS18-like nucleic acids/genes and variants thereof may be suitable in practising the methods of the invention. Variant OsMADS18-like nucleic acid/genes include portions of an OsMADS18-like nucleic acid/gene and/or nucleic acids capable of hybridising with an OsMADS18-like nucleic acid/gene.

The term portion as defined herein refers to a piece of DNA (an OsMADS18-like nucleic acid/gene) comprising at least 747 nucleotides which portion encodes a polypeptide of at least 249 amino acids, which polypeptide comprises at least features (i) and (ii) as follow, preferably together with feature (iii) and/or feature (iv), features (i) to (iv) being: (i) a monocot type 11 MADS-box transcription factor of the SQUAMOSA clade; (ii) having DNA and protein binding activity; and (iii) comprising the motif LPPWMLRT (SEQ ID NO: 18) in the last 15 amino acids at the C terminus of the protein, allowing for one amino acid substitution anywhere in the motif but not in the second and fourth positions of the motif, which are respectively a proline and a tryptophan; and (iv) having at least 65% sequence identity to the OsMADS18 protein of SEQ ID NO: 2. A portion may be prepared, for example, by making one or more deletions to an OsMADS18-like nucleic acid. The portions may be used in isolated form or they may be fused to other coding (or non coding) sequences in order to, for example, produce a protein that combines several activities. When fused to other coding sequences, the resulting polypeptide produced upon translation may be bigger than that predicted for the OsMADS18-like fragment. Preferably, the functional portion is a portion of a nucleic acid as represented by any one of SEQ ID NO: 1, SEQ ID NO: 3, SEQ ID NO: 5, SEQ ID NO: 7, SEQ ID NO: 14 and SEQ ID NO: 16.

Another variant of an OsMADS18-like nucleic acid/gene is a nucleic acid capable of hybridising under reduced stringency conditions, preferably under stringent conditions, with an OsMADS18-like nucleic acid/gene as hereinbefore defined, which hybridising sequence encodes a polypeptide comprising at least features (i) and (ii) as follow, preferably together with feature (iii) and/or feature (iv), features (i) to (iv) being: (i) a monocot type II MADS-box transcription factor of the SQUAMOSA clade; and (ii) a polypeptide having DNA and protein binding activity; and (iii) a polypeptide comprising the motif LPPWMLRT (SEQ ID NO: 18) located in the last 15 amino acids of the protein, allowing for one amino acid substitution anywhere in the motif but not in the second and fourth positions of the motif, which are respectively a proline and a tryptophan; and (iv) having at least 65% sequence identity to the OsMADS18 protein of SEQ ID NO: 2. In addition to having the aforementioned features, preferably the hybridising sequence is one that is capable of hybridising to a nucleic acid as represented by any one of SEQ ID NO: 1, SEQ ID NO: 3, SEQ ID NO: 5, SEQ ID NO: 7, SEQ ID NO: 14, and SEQ ID NO: 16, or to a portion of any of the aforementioned sequences as defined hereinabove.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” in the context of nucleic acid hybridisation experiments such as Southern and Northern hybridisations are sequence dependent and differ under different environmental parameters. The skilled artisan is aware of various parameters which may be altered during hybridisation and washing to either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M. Formamide reduces the melting temperature of DNA-DNA and DNA-RNA duplexes with 0.6 to 0.7° C. for each percent formamide, and addition of 50% formamide allows hybridisaton to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tₘ may be calculated using the following equations, depending on the types of hybrids:
1. DNA-DNA hybrids (Meinkoth and Wahl, Anal. Biochem., 138: 267-284, 1984):
   Tₘ=81.5° C.+16.6×log[Na⁺]^{a}+0.41×%[G/C^{b}]−500×[L^{c}]⁻¹−0.61×% formamide
2. DNA-RNA or RNA-RNA hybrids:
   Tₘ=79.8+18.5 (log₁₀[Na⁺]^{a})+0.58 (%G/C^{b})+11.8 (%G/C^{b})²−820/L^{c}
3. oligo-DNA or oligo-RNAd hybrids:
   For <20 nucleotides: Tₘ=2 (Iₙ)
   For 20-35 nucleotides: Tₘ=22+1.46 (Iₙ)
^{a} or for other monovalent cation, but only accurate in the 0.01-0.4 M range.
^{b} only accurate for %GC in the 30% to 75% range.
^{c} L=length of duplex in base pairs.
^{d} Oligo, oligonucleotide; Iₙ, effective length of primer=2×(no. of G/C)+(no. of A/T).

Note: for each 1% formamide, the Tₘ is reduced by about 0.6 to 0.7° C., while the presence of 6 M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. Conditions of greater or less stringency may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein containing solutions, additions of heterologous RNA, DNA, and SDS to the hybridisation buffer, and treatment with RNase. Examples of hybridisation and wash conditions are listed in Table 3 below.

TABLE 3
Examples of hybridisation and wash conditions

| | | | | Wash |
|---|---|---|---|---|
| Stringency | Polynucleotide | Hybrid | Hybridization Temprerature | Temperature |
| Condition | Hybrid ^{±} | Length (bp) ^{‡} | and Buffer^{†} | and Buffer^{†} |
| A | DNA:DNA | > or | 65° C. 1 × SSC; or 42° C., 1 × SSC | 65° C.; |
| | | equal to 50 | and 50% formamide | 0.3 × SSC |
| B | DNA:DNA | <50 | Tb*; 1 × SSC | Tb*; 1 × SSC |
| C | DNA:RNA | > or | 67° C. 1 × SSC; or 45° C., 1 × SSC | 67° C.; |
| | | equal to 50 | and 50% formamide | 0.3 × SSC |
| D | DNA:RNA | <50 | Td*; 1 × SSC | Td*; 1 × SSC |
| E | RNA:RNA | > or | 70° C. 1 × SSC; or 50° C., 1 × SSC | 70° C.; |
| | | equal to 50 | and 50% formamide | 0.3 × SSC |
| F | RNA:RNA | <50 | Tf*; 1 × SSC | Tf*; 1 × SSC |
| G | DNA:DNA | > or | 65° C. 4 × SSC; or 45° C., 4 × SSC | 65° C.; 1 × SSC |
| | | equal to 50 | and 50% formamide | |
| H | DNA:DNA | <50 | Th*; 4 × SSC | Th*; 4 × SSC |
| I | DNA:RNA | > or | 67° C. 4 × SSC; or 45° C., 4 × SSC | 67° C.; 1 × SSC |
| | | equal to 50 | and 50% formamide | |
| J | DNA:RNA | <50 | Tj*; 4 × SSC | Tj*; 4 × SSC |
| K | RNA:RNA | > or | 70° C. 4 × SSC; or 40° C., 6 × SSC | 67° C.; 1 × SSC |
| | | equal to 50 | and 50% formamide | |
| L | RNA:RNA | <50 | Tl*; 2 × SSC | Tl*; 2 × SSC |
| M | DNA:DNA | > or | 50° C. 4 × SSC; or 40° C., 6 × SSC | 50° C.; 2 × SSC |
| | | equal to 50 | and 50% formamide | |
| N | DNA:DNA | <50 | Tn*; 6 × SSC | Tn*; 6 × SSC |
| O | DNA:RNA | > or | 55° C. 4 × SSC; or 42° C., 6 × SSC | 55° C.; 2 × SSC |
| | | equal to 50 | and 50% formamide | |
| P | DNA:RNA | <50 | Tp*; 6 × SSC | Tp*; 6 × SSC |
| Q | RNA:RNA | > or | 60° C. 4 × SSC; or 45° C., 6 × SSC | 60° C.; |
| | | equal to 50 | and 50% formamide | 2 × SSC |
| R | RNA:RNA | <50 | Tr*; 4 × SSC | Tr*; 4 × SSC |
| ^{‡}The “hybrid length” is the anticipated length for the hybridising nucleic acid. When nucleic acids of known sequence are hybridised, the hybrid length may be determined by aligning the sequences and identifying the conserved regions described herein. | | | | |
| ^{†}SSPE (1 × SSPE is 0.15 M NaCl, 10 mM NaH₂PO₄, and 1.25 mM EDTA, pH 7.4) may be substituted for SSC (1 × SSC is 0.15 M NaCl and 15 mM sodium citrate) in the hybridisation and wash buffers; washes are performed for 15 minutes after hybridisation is complete. The hybridisations and washes may additionally include 5 × Denhardt's reagent, 0.5-1.0% SDS, 100 μg/ml denatured, fragmented salmon sperm DNA, 0.5% sodium pyrophosphate, and up to 50% | | | | |
| # formamide. | | | | |

*Tb-Tr: The hybridisation temperature for hybrids anticipated to be less than 50 base pairs in length should be 5-10° C. less than the melting temperature Tₘ of the hybrids; the Tₘ is determined according to the abovementioned equations.
^{±}The present invention also encompasses the substitution of any one, or more DNA or RNA hybrid partners with either a PNA, or a modified nucleic acid.

For the purposes of defining the level of stringency, reference can be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

The OsMADS18-like nucleic acid or variant thereof may be derived from any natural or artificial source. The nucleic acid/gene or variant thereof may be isolated from a microbial source, such as yeast or fungi, or from a plant, algae or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a monocotyledonous species, preferably from the family Poaceae, further preferably from Oryza sativa. More preferably, the OsMADS18-like nucleic acid isolated from Oryza sativa is represented by SEQ ID NO: 1 and the OsMADS18-like amino acid sequence is as represented by SEQ ID NO: 2.

The activity of an OsMADS18-like polypeptide or a homologue thereof may be increased by introducing a genetic modification (preferably in the locus of an OsMADS18-like gene). The locus of a gene as defined herein is taken to mean a genomic region, which includes the gene of interest and 10 kb up- or down stream of the coding region.

The genetic modification may be introduced, for example, by any one (or more) of the following methods: T-DNA activation, TILLING, site-directed mutagenesis, directed evolution, homologous recombination or by introducing and expressing in a plant a nucleic acid encoding an OsMADS18-like polypeptide or a homologue thereof. Following introduction of the genetic modification, there follows a step of selecting for increased activity of an OsMADS18-like polypeptide, which increase in activity gives plants having increased yield.

T-DNA activation tagging (Hayashi et aL Science (1992) 1350-1353) involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-preferred, cell type-preferred and inducible promoters are all suitable for use in T-DNA activation.

A genetic modification may also be introduced in the locus of an OsMADS18-like gene using the technique of TILLING (Targeted Induced Local Lesions In Genomes). This mutagenesis technology is useful to generate, identify and isolate mutagenised variants of an OsMADS18-like nucleic acid capable of exhibiting OsMADS18-like activity. TILLING also allows selection of plants carrying such mutant variants. These mutant variants may even exhibit higher OsMADS18-like activity than that exhibited by the gene in its natural form. TILLNG combines high-density mutagenesis with high-throughput screening methods. The steps typically followed in TILLING are: (a) EMS mutagenesis (Redei GP and Koncz C (1992) In Methods in Arabidopsis Research, Koncz C, Chua N H, Schell J. eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

Site-directed mutagenesis may be used to generate variants of OsMADS18-like nucleic acids. Several methods are available to achieve site-directed mutagenesis, the most common being PCR based methods (current protocols in molecular biology. Wiley Eds. http://www.4ulr.com/products/currentprotocols/index.html).

Directed evolution may also be used to generate variants of OsMADS18-like nucleic acids. This consists of iterations of DNA shuffling followed by appropriate screening and/or selection to generate variants of OsMADS18-like nucleic acids or variants thereof encoding OsMADS18-like polypeptides or homologues thereof having a modified biological activity (Castle et aL, (2004) Science 304(5674): 1151-4; U.S. Pat. Nos. 5,811,238 and 6,395,547).

T-DNA activation, TILLING, site-directed mutagenesis and directed evolution are examples of technologies that enable the generation of novel alleles and OsMADS18-like variants. Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; lida and Terada (2004) Curr Opin Biotech 15(2):132-8). The nucleic acid to be targeted (which may be an OsMADS18-like nucleic acid or variant thereof as hereinbefore defined) need not be targeted to the locus of an OsMADS18-like gene, but may be introduced in, for example, regions of high expression. The nucleic acid to be targeted may be an improved allele used to replace the endogenous gene or may be introduced in addition to the endogenous gene.

According to a preferred embodiment of the invention, plant yield may be improved by introducing and expressing in a plant a nucleic acid encoding an OsMADS18-like polypeptide or a homologue thereof.

A preferred method for introducing a genetic modification (which in this case need not be in the locus of an OsMADS18-like gene) is to introduce and express in a plant a nucleic acid encoding an OsMADS18-like polypeptide or a homologue thereof. An OsMADS18-like polypeptide or a homologue thereof as mentioned above is (i) a monocot type 11 MADS-box transcription factor of the SQUAMOSA lade; and (ii) has DNA and protein binding activity; and (iii) comprises the motif LPPWMLRT (SEQ ID NO: 18) located in the last 15 amino acids of the protein, allowing for one amino acid substitution anywhere in the motif but not in the second and fourth positions of the motif, which are respectively a proline and a tryptophan; and (iv) has at least 65 % sequence identity to the OsMADS18 protein of SEQ ID NO: 2. The nucleic acid to be introduced into a plant may be a full-length nucleic acid or may be a portion or a hybridizing sequence as hereinbefore defined.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company and Table 1 above).

Also encompassed by the term “homologues” are two special forms of homology, which include orthologous sequences and paralogous sequences, which encompass evolutionary concepts used to describe ancestral relationships of genes. The term “paralogous” relates to gene-duplications within the genome of a species leading to paralogous genes. The term “orthologous” relates to homologous genes in different organisms due to speciation.

Orthologues in, for example, other monocot plant species may easily be found by performing a so-called reciprocal blast search. This may be done by a first blast involving blasting a query sequence (for example, SEQ ID NO: 1 or SEQ ID NO: 2) against any sequence database, such as the publicly available NCBI database which may be found at: http://www.ncbi.nim.nih.gov. BLASTn or tBLASTX (using standard default values) may be used when starting from nucleotide sequence and BLASTP or TBLASTN (using standard default values) may be used when starting from a protein sequence. The BLAST results may optionally be filtered. The sequences of either the filtered or non-filtered results are then BLASTed back (second BLAST) against the sequences from the same organism as the query sequence organism, (where the query sequence is SEQ ID NO: 1 or SEQ ID NO: 2 the second blast would therefore be against Oryza sativa (rice) sequences). The results of the first and second BLASTs are then compared. A paralogue is identified if a high-ranking hit from the second blast is from the same species as from which the query sequence is derived; an orthologue is identified if a high-ranking hit is not from the same species as from which the query sequence is derived. High-ranking hits are those having a low E-value. The lower the E-value, the more significant the score (or in other words the lower the chance that the hit was found by chance). Computation of the E-value is well known in the art. In the case of large families, ClustalW may be used, followed by a neighbour joining tree, to help visualize clustering of related genes and to identify orthologues and paralogues.

A homologue may be in the form of a “substitutional variant” of a protein, i.e. where at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions.

A homologue may also be in the form of an “insertional variant” of a protein, i.e. where one or more amino acid residues are introduced into a predetermined site in a protein. Insertions may comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

Homologues in the form of “deletion variants” of a protein are characterised by the removal of one or more amino acids from a protein.

Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The OsMADS18-like polypeptide or homologue thereof may be a derivative. “Derivatives” include peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as presented in SEQ ID NO: 2. “Derivatives” of a protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The OsMADS18-like polypeptide or homologue thereof may be encoded by an alternative splice variant of an OsMADS18-like nucleic acid/gene. The term “alternative splice variant” as used herein encompasses variants of a nucleic acid sequence in which selected introns and/or exons have been excised, replaced or added, or in which introns have been shortened or lengthened. Such variants will be ones in which the biological activity of the protein is retained, which may be achieved by selectively retaining functional segments of the protein. Such splice variants may be found in nature or may be manmade. Methods for making such splice variants are well known in the art. Preferred splice variants are splice variants of the nucleic acid represented by SEQ ID NO: 1, SEQ ID NO: 3, SEQ ID NO: 5, SEQ ID NO: 7, SEQ ID NO: 14 and SEQ ID NO: 16. Further preferred are splice variants encoding a polypeptide comprising at least features (i) and (ii) as follow, preferably together with feature (iii) and/or feature (iv), features (i) to (iv) being (i) a monocot type II MADS-box transcription factor of the SQUAMOSA lade; and (ii) having DNA and protein binding activity; and (iii) comprising the motif LPPWMLRT (SEQ ID NO: 18) located in the last 15 amino acids of the protein, allowing for one amino acid substitution anywhere in the motif but not except in the second and fourth positions of the motif, which are respectively a proline and a tryptophan; and (iv) having at least 65% sequence identity to the OsMADS18 protein of SEQ ID NO: 2.

The homologue may also be encoded by an allelic variant of a nucleic acid encoding an OsMADS18-like polypeptide or a homologue thereof, preferably an allelic variant of a nucleic acid represented by SEQ ID NO: 1, SEQ ID NO: 3, SEQ ID NO: 5, SEQ ID NO: 7, SEQ ID NO: 14 or SEQ ID NO: 16. Further preferably, the polypeptide encoded by the allelic variant comprises at least features (i) and (ii) as follow, preferably together with feature (iii) and/or feature (iv), features (i) to (iv) being: (i) a monocot type II MADS-box transcription factor of the SQUAMOSA lade; and (ii) having DNA and protein binding activity; and (iii) comprising the motif LPPWMLRT (SEQ ID NO: 18) located in the last 15 amino acids of the protein, allowing for one amino acid substitution anywhere in the motif but not in the second and fourth positions of the motif, which are respectively a proline and a tryptophan; and (iv) having at least 65% sequence identity to the OsMADS18 protein of SEQ ID NO: 2. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

According to a preferred aspect of the present invention, enhanced or increased expression of the OsMADS18-like nucleic acid or variant thereof is envisaged. Methods for obtaining enhanced or increased expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of an OsMADS18-like nucleic acid or variant thereof. For example, endogenous promoters may be altered in vivo by mutation, deletion or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold, Buchman and Berg, Mol. Cell biol. 8:4395-4405 (1988); Callis et al., Genes Dev. 1:1183-1200 (1987). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention so as to give plants having increased yield relative to corresponding wild type plants.

Therefore, there is provided a gene construct comprising:
(i) An OsMADS18-like nucleic acid or variant thereof;
(ii) One or more control sequences capable of driving expression of the nucleic acid sequence of (i); and optionally
(iii) A transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells.

Plants are transformed with a vector comprising the sequence of interest (i.e., an OsMADS18-like nucleic acid or variant thereof). The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a—35 box sequence and/or—10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

The OsMADS18-like nucleic acid or variant thereof is preferably operably linked to an early shoot apical meristem promoter. An “early shoot apical meristem promoter” as defined herein is a promoter that is transcriptionally active in the shoot apical meristem from the embryo globular stage up to the young seedling stage; these stages being well known to persons skilled in the art. Preferably, the early shoot apical meristem promoter is an OSH1 promoter (from rice; SEQ ID NO: 22 (Matsuoka et al., (1993) Plant Cell 5: 1039-1048; Sato et al., (1996) Proc Natl Acad Sci U S A 93(15): 8117-22). It should be clear that the applicability of the present invention is not restricted to the OsMADS18-like nucleic acid represented by SEQ ID NO: 1, nor is the applicability of the invention restricted to expression of an OsMADS18-like nucleic acid when driven by an OSH1 promoter. Examples of other early shoot apical meristem promoters are shown in Table 4 below. These are members of the KNOX family class 1 homeobox, from paralogous or orthologous genes. It should be understood that the list below is non-exhaustive.

TABLE 4
Examples of early apical meristem promoters

| Gene | | | |
|---|---|---|---|
| source | Gene family | Plant source | Reference |
| OSH1 | KNOX family class 1 | Oryza sativa | Matsuoka et al., (1993) Plant Cell 5: |
| | homeobox | | 1039-1048 |
| | | | Sato et al., (1996) PNAS 93: 8117- |
| | | | 8122 |
| Knotted1 | KNOX family class 1 | Zea mays | Hake et al., (1989) EMBO Journal 8: |
| | homeobox | | 15-22 |
| KNAT1 | KNOX family class 1 | Arabidopsis | Lincoln et al., (1994) Plant Cell 6: |
| | homeobox | thaliana | 1859-1876 |
| Oskn2 | KNOX family class 1 | Oryza sativa | Postma-Haarsma et al., (1999) Plant |
| | homeobox | | Mol Biol 39(2): 257-71 |
| Oskn3 | KNOX family class 1 | Oryza sativa | Postma-Haarsma et al., (1999) Plant |
| | homeobox | | Mol Biol 39(2): 257-71 |

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the fl-ori and colEl.

The genetic construct may optionally comprise a selectable marker gene. As used herein, the term “selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptil that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

The present invention also encompasses plants obtainable by the methods according to the present invention. The present invention therefore provides plants obtainable by the methods according to the present invention, which plants have introduced therein an OsMADS18-like nucleic acid or variant thereof.

The invention also provides a method for the production of transgenic plants having increased yield relative to corresponding wild type plants, comprising introduction and expression in a plant of an OsMADS18-like nucleic acid or a variant thereof.

More specifically, the present invention provides a method for the production of transgenic plants having increased yield relative to corresponding wild type plants, which method comprises:
(i) introducing and expressing in a plant, plant part or plant cell an OsMADS18-like nucleic acid or variant thereof; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al., 1985 Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic rice plants expressing an OsMADS18-like nucleic acid/gene are preferably produced via Agrobacterium-mediated transformation using any of the well known methods for rice transformation, such as described in any of the following: published European patent application EP1198985 A1; Aldemita and Hodges (Planta 199: 612-617, 1996); Chan et al. (Plant Mol Biol 22 (3): 491-506, 1993); Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention. The invention also includes host cells containing an isolated OsMADS18-like nucleic acid or variant thereof. Preferred host cells according to the invention are plant cells. The invention also extends to harvestable parts of a plant such as, but not limited to seeds, leaves, fruits, flowers, stem cultures, rhizomes, tubers and bulbs. The invention furthermore relates to products derived from a harvestable part of such a plant, such as dry pellets or powders, oil, fat and fatty acids, starch or proteins.

The present invention also encompasses use of OsMADS18-like nucleic acids or variants thereof and use of OsMADS18-like polypeptides or homologues thereof.

One such use relates to improving plant yield relative to corresponding wild type plants, in particular in improving seed yield. The increase in yield being as defined hereinabove.

OsMADS18-like nucleic acids or variants thereof, or OsMADS18-like polypeptides or homologues thereof may find use in breeding programmes in which a DNA marker is identified which may be genetically linked to an OsMADS18-like gene or variant thereof. The OsMADS18-like nucleic acids/ genes or variants thereof, or OsMADS18-like polypeptides or homologues thereof may be used to define a molecular marker. This DNA or protein marker may then be used in breeding programmes to select plants having increased yield. The OsMADS18-like gene or variant thereof may, for example, be a nucleic acid as represented by any one of SEQ ID NO: 1, SEQ ID NO: 3, SEQ ID NO: 5, SEQ ID NO: 7, SEQ ID NO: 14, and SEQ ID NO: 16.

Allelic variants of an OsMADS18-like nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring yield performance of plants containing different allelic variants of the sequence in question, for example, different allelic variants of any one of SEQ ID NO: 1, SEQ ID NO: 3, SEQ ID NO: 5, SEQ ID NO: 7, SEQ ID NO: 14, and SEQ ID NO: 16. The yield performance may be monitored in a greenhouse or in the field. Further optional steps include crossing plants, in which the superior allelic variant was identified, with another plant. This could be used, for example, to make a combination of interesting phenotypic features.

An OsMADS18-like nucleic acid or variant thereof may also be used as probes for genetically and physically mapping the genes that they are a part of, and as markers for traits linked to those genes. Such information may be useful in plant breeding in order to develop lines with desired phenotypes. Such use of OsMADS18-like nucleic acids or variants thereof requires only a nucleic acid sequence of at least 15 nucleotides in length. The OsMADS18-like nucleic acids or variants thereof may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the OsMADS18-like nucleic acids or variants thereof. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the OsMADS18-like nucleic acid or variant thereof in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et aL In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11 :95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield, as described hereinbefore. The trait of increased yield may also be combined with other economically advantageous traits, such as further yield-enhancing traits, tolerance to various stresses, traits modifying various architectural features and/or biochemical and/or physiological features.

### DESCRIPTION OF FIGURES

The present invention will now be described with reference to the following figures in which:

FIG. 1 shows the typical domain structure of MIKC MADS box transcription factors. The MADS domain is located at the amino-terminal end of the protein and encodes a DNA binding and dimerization function. The conserved K domain is involved in protein dimerisation. The I and the C domains are less well conserved. The C domain can be involved in transcriptional activation or in the formation of higher-order MADS multimers (from Jack (2001) Plant Molec Biol 46: 515-520).

FIG. 2 shows a multiple alignment of several plant MADS domain transcription factors of the SQUAMOSA clade, using VNTI AlignX multiple alignment program, based on a modified ClustalW algorithm (InforMax, Bethesda, MD, http://www.informaxinc.com), with default settings for gap opening penalty of 10 and a gap extension of 0.05). The SQUAMOSA-specific residues of the MADS domain are boxed across the alignment including the MADS consensus sequence. The two dicot subclades within the SQUAMOSA dade are indicated as dicot AP1 dade and dicot FUL lade. The monocot sequences are further subdivided with respect to their conserved motifs in the C-terminal end of the protein, LPPWMLRT and LPPWMLSH that are boxed in bold.

FIG. 3 represents an unrooted neighbour joining tree, derived from a sequence alignment using ClustalW 1.83 with default values. The two major groups, comprising monocots and dicots proteins, can each be further split up into AP1 and FUL subclades for the dicots, and C-terminal-comprising motifs LPPWMLRT (SEQ ID NO: 18) and LPPWMLSH (SEQ ID NO: 19) for the monocots. The source, description and accession number of the polypeptide sequences used are given in the table below.

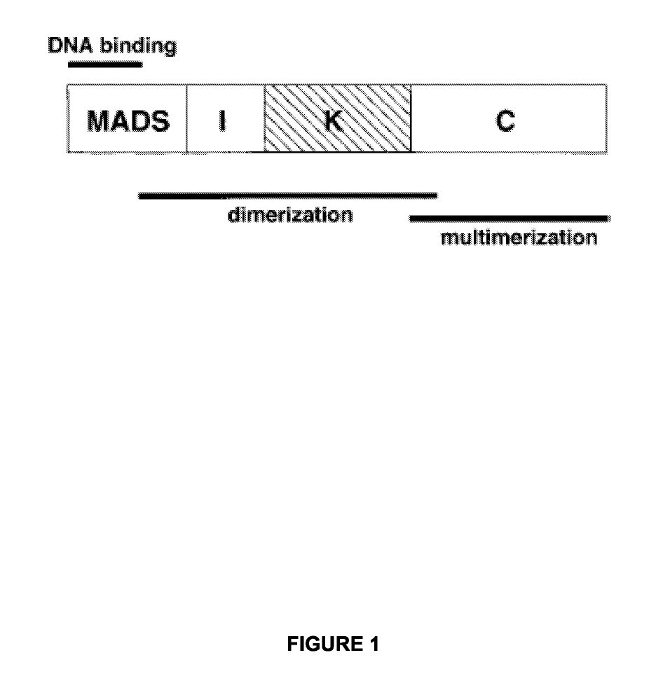

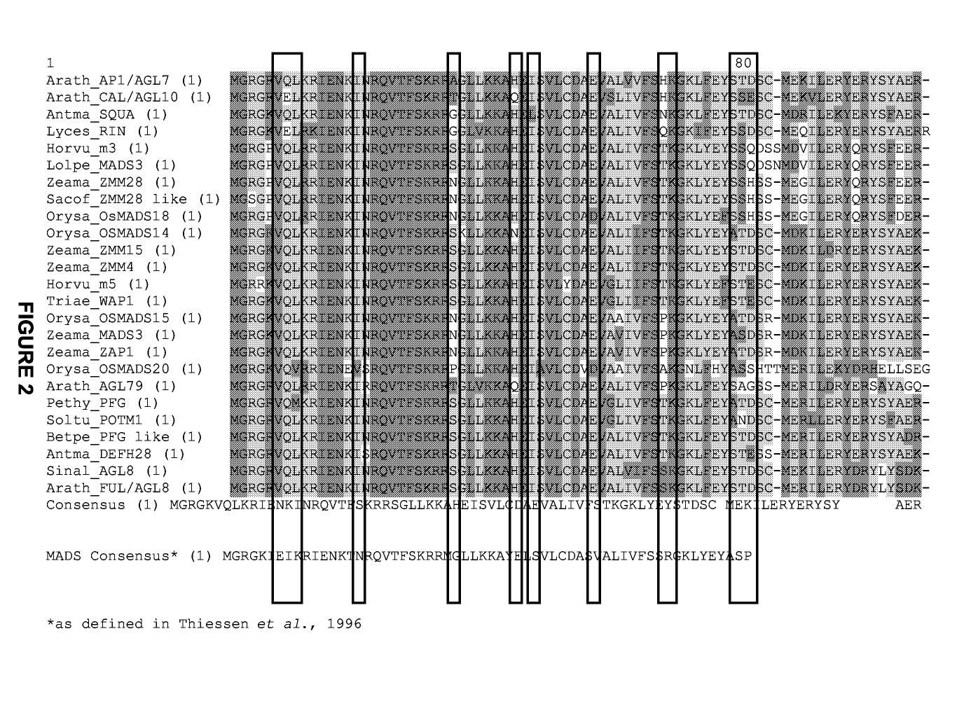

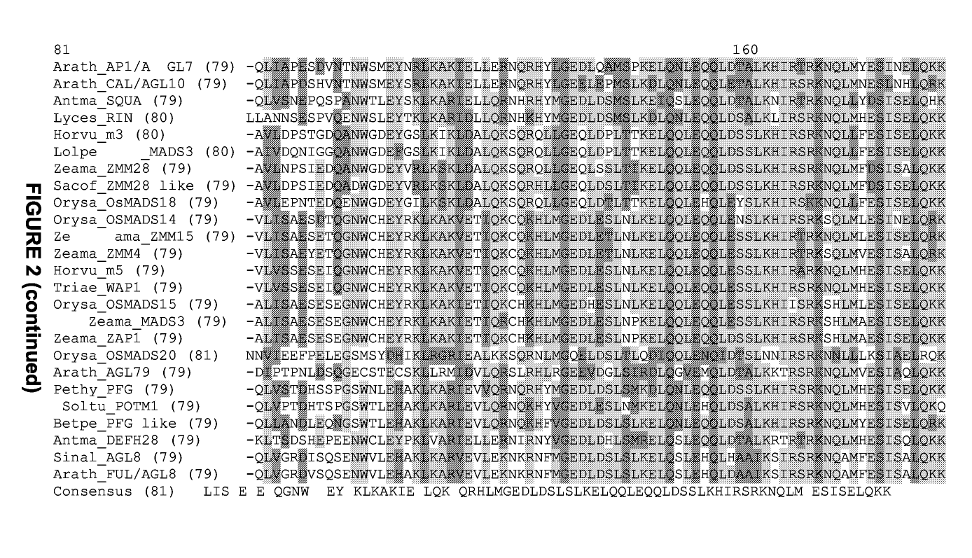

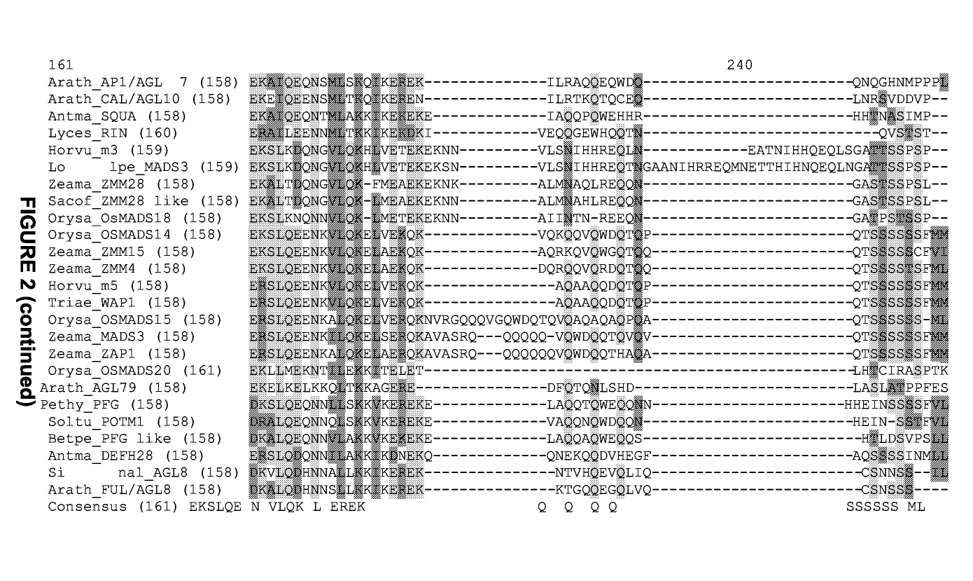

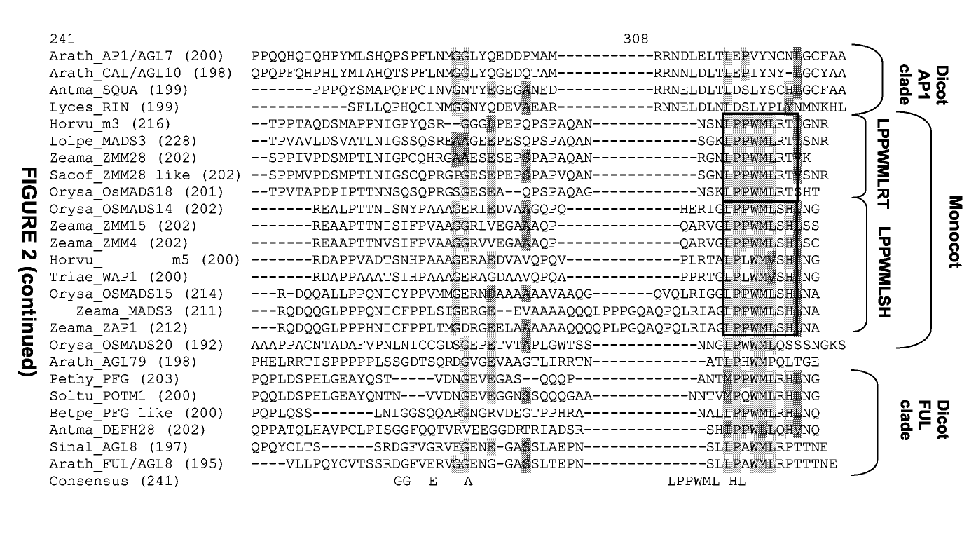

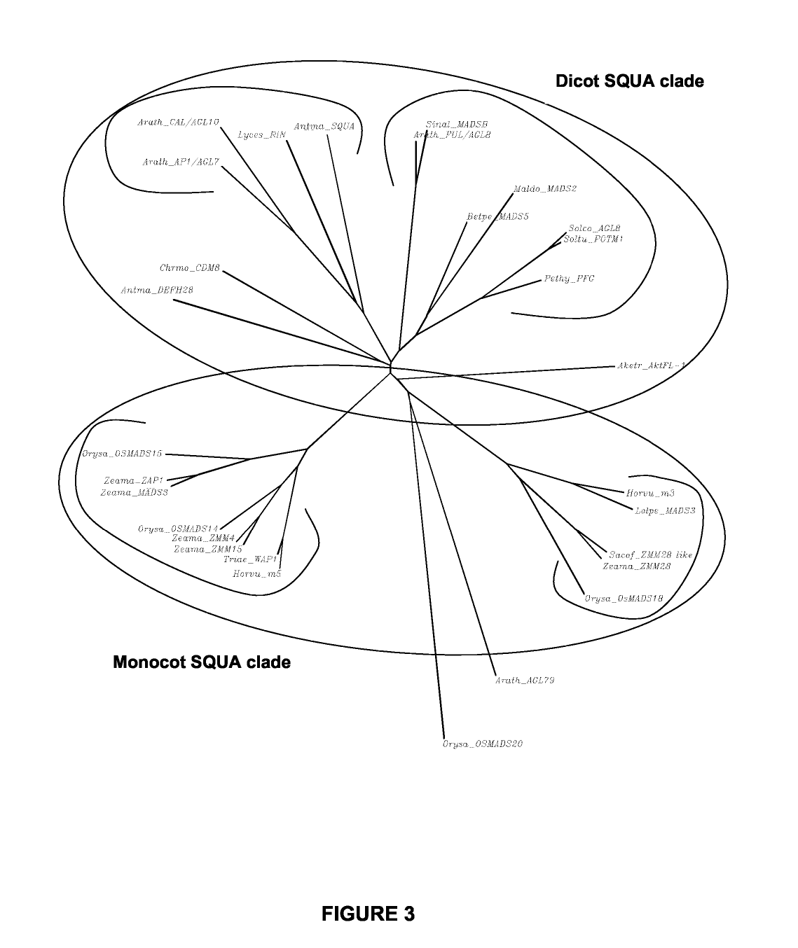

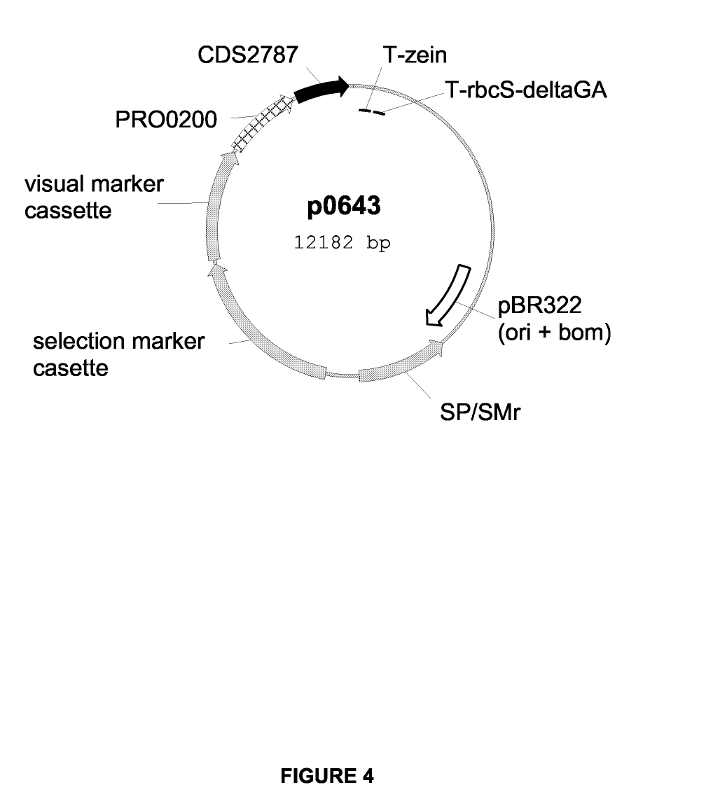

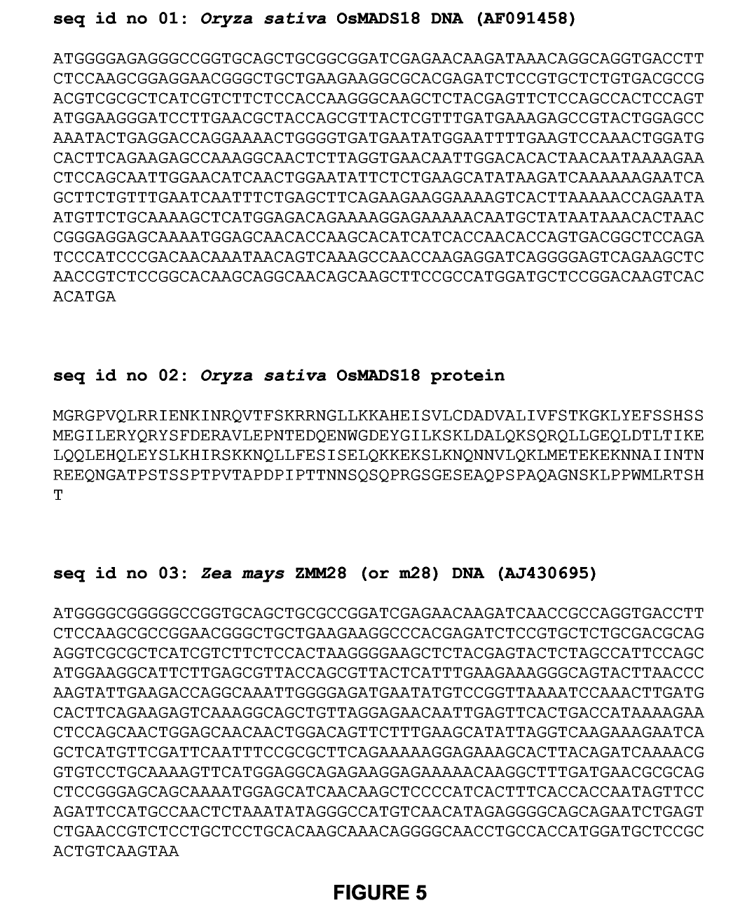

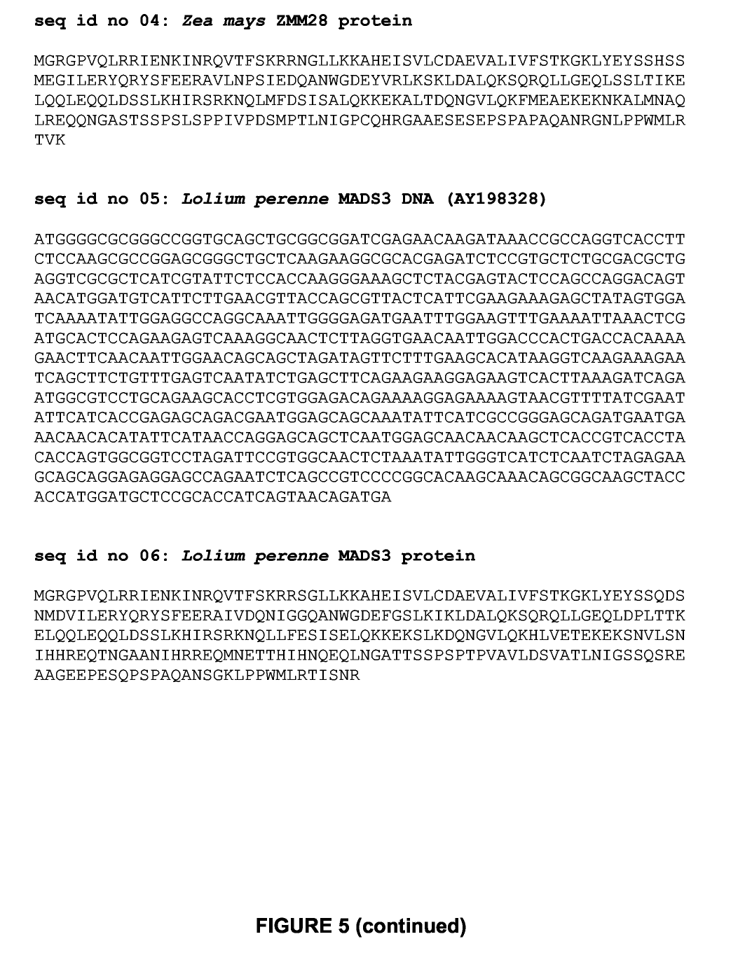

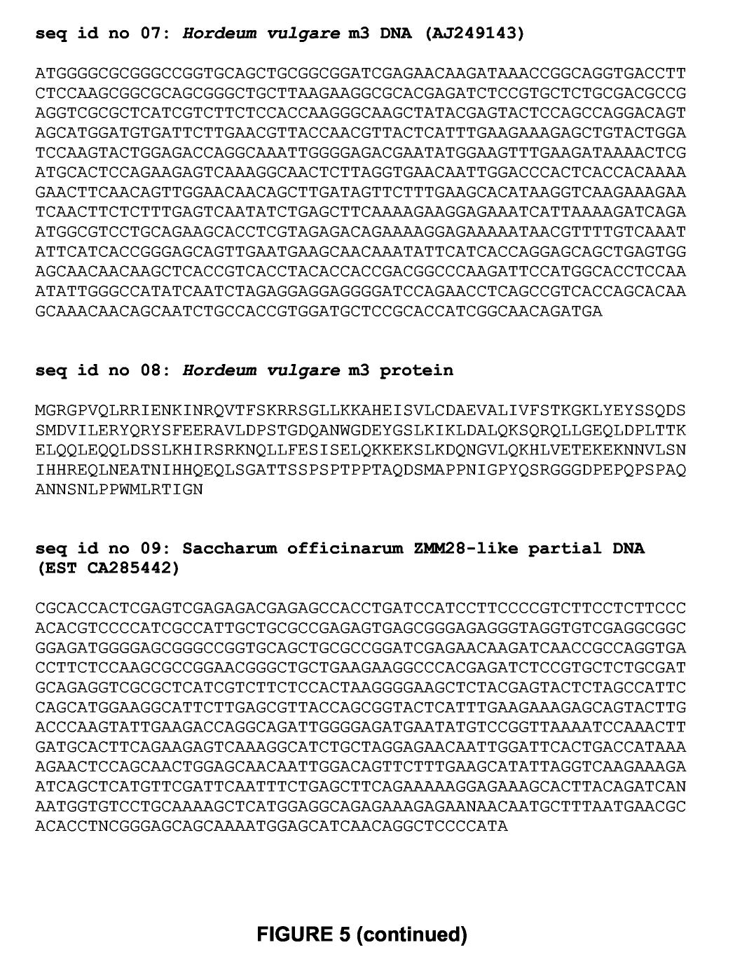

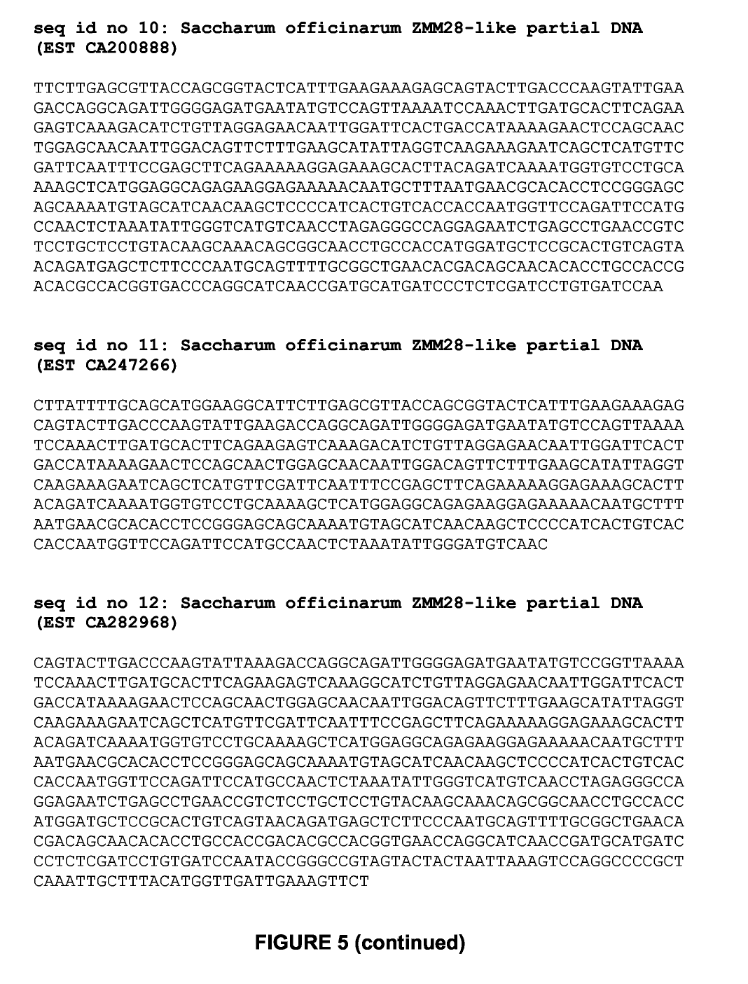

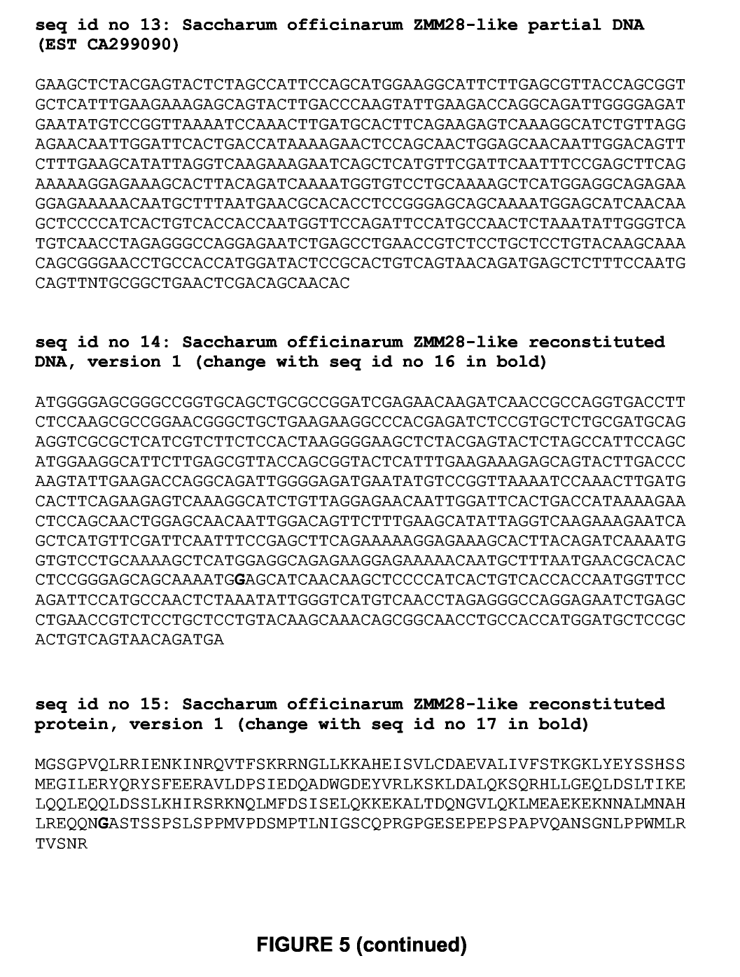

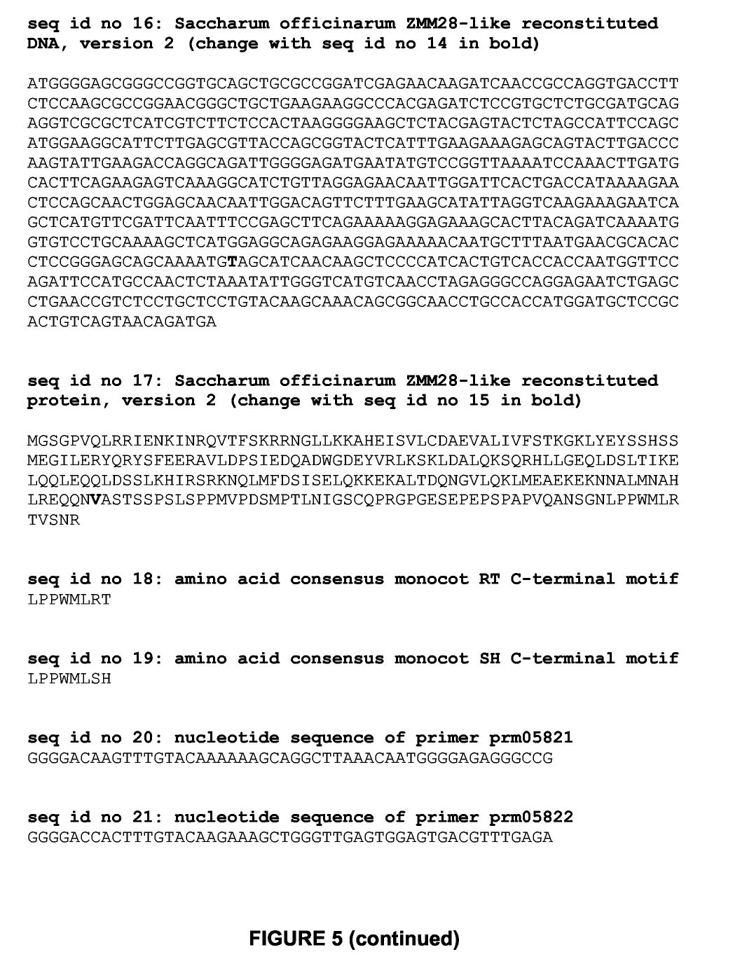

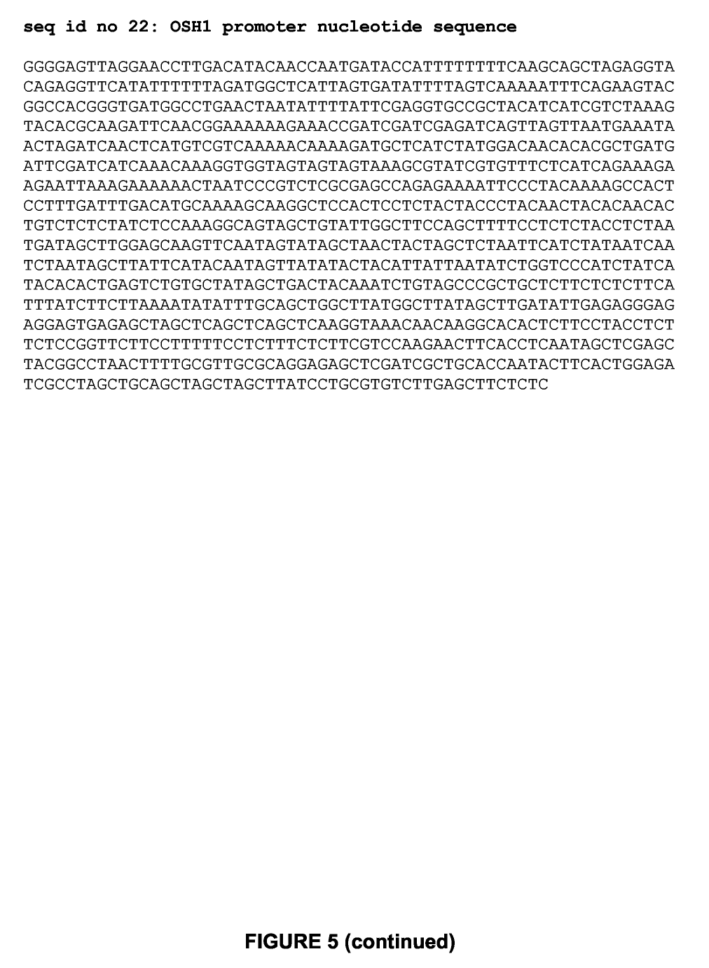

| | | NCBI accession |
|---|---|---|
| Source | Description | number |
| Akebia trifoliata | FRUITFULL-like protein; Aketr_AktFL-1 | AAT46099 |
| Antirrhinum majus | DEFH28; Antma_DEFH28 | AAK72467 |
| Antirrhinum majus | SQUA; Antma_SQUA | CAA45228 |
| Arabidopsis thaliana | MADS-box protein AGL79; Arath_AGL79 | AAN52802 |
| Arabidopsis thaliana | APETALA1/AGL7; Arath_AP1/AGL7 | P35631 |
| Arabidopsis thaliana | CAL/AGL10; Arath_CAL/AGL10 | Q39081 |
| Arabidopsis thaliana | FUL/AGL8; Arath_FUL/AGL8 | Q38876 |
| Betula pendula | MADS5; Betpe_MADS5 | CAA67969 |
| Chrysanthemum x morifolium | CDM8; Chrmo_CDM8 | AAO22981 |
| Hordeum vulgare | MADS-box protein 3; Horvu_m3 | CAB97351 |
| Hordeum vulgare | MADS-box protein 5; Horvu_m5 | CAB97352 |
| Lolium perenne | MADS3; Lolpe_MADS3 | AAO45875 |
| Lycopersicum esculentum | RIN; Lyce_RIN | AAM15775 |
| Malus X domestica | MADS2; Maldo_MADS2 | AAC83170 |
| Oryza sativa | OsMADS14; Orysa_OsMADS14 | AAF19047 |
| Oryza sativa | OsMADS15; Orysa_OsMADS15 | AAF19048 |
| Oryza sativa | OsMADS18; Orysa_OsMADS18 | AAF04972 |
| Oryza sativa | OsMADS20; Orysa_OsMADS20 | AA092341 |
| Petunia hybrida | PFG; Pethy_PFG | AAQ72509 |
| Saccharum officinarum | ZMM28-like Sacof_ZMM28 like | SEQ ID NO:15, 17 |
| Sinapis alba | MADS B; Sinal_MADSB | Q41274 |
| Solanum commersonii | AGL8 homolog; Solco_AGL8 | O22328 |
| Solanum tuberosum | POTM1-1; Soltu_POTM1-1 | Q42429 |
| Triticum aestivum | WAP1; Triae_WAP1 | BAA33457 |
| Zea mays | MADS3; Zeama_MADS3 | AAG43200 |
| Zea mays | ZAP1; Zeama_ZAP1 | AAB00081 |
| Zea mays | ZMM15; Zeama_ZMM15 | CAD23408 |
| Zea mays | ZMM28; Zeama_ZMM28 | CAD23441 |
| Zea mays | ZMM4; Zeama_ZMM4 | CAD23417 |

FIG. 4 shows a binary vector p0643, for expression in Oryza sativa of an Oryza sativa OsMADS18-like (internal reference CDS2787) under the control of an OSH1 promoter (internal reference PRO0200).

FIG. 5 details examples of sequences useful in performing the methods according to the present invention.

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, published by BIOS Scientific Publications Ltd (UK) and Blackwell Scientific Publications (UK).

### Example 1

### Gene Cloning

The Oryza sativa OsMADS18-like gene (CDS2787) was amplified by PCR using as template an Oryza sativa seedling cDNA library (Invitrogen, Paisley, UK). After reverse transcription of RNA extracted from seedlings, the cDNAs were cloned into pCMV Sport 6.0. Average insert size of the bank was 1.6 kb and the original number of clones was of the order of 1.67×10⁷ cfu. Original titer was determined to be 3.34×10⁶ cfu/ml after first amplification of 6×10¹⁰ cfu/ml. After plasmid extraction, 200 ng of template was used in a 50 μl PCR mix. Primers prm05821 (SEQ ID NO: 20; sense, start codon in bold, AttB1 site in italic: 5′-GGGGACAAGTTTGTACAAAAAAGCAGGCTTCACAATGGGGAGAGGGCCG 3′) and prm05822 (SEQ ID NO: 21; reverse, complementary, AttB2 site in italic: 5′ GGGGACCACTTTGTACAAGAAAGCTGGGTTGAGTGGAGTGACGTTTGAGA3′), which include the AttB sites for Gateway recombination, were used for PCR amplification. PCR was performed using Hifi Taq DNA polymerase in standard conditions. A PCR fragment of 849 bp (including attB sites) was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, p05838. Plasmid pDONR201 was purchased from lnvitrogen, as part of the Gateway® technology.

### Example 2

### Vector Construction

The entry clone p05838 was subsequently used in an LR reaction with p05294, a destination vector used for Oryza sativa transformation. This vector contains as functional elements within the T-DNA borders: a plant selectable marker; a screenable marker expression cassette; and a Gateway cassette intended for LR in vivo recombination with the sequence of interest already cloned in the entry clone. A rice OSH1 promoter (SEQ ID NO 22) for early apical meristem expression (PRO0200) was located upstream of this Gateway cassette (Matsuoka et aL, Plant Cell 5 1993,1039-1048).

After the LR recombination step, the resulting expression vector p0643 (FIG. 4) was transformed into Agrobacterium strain LBA4044 and subsequently to Oryza sativa plants. Transformed rice plants were allowed to grow and were then examined for the parameters described in Example 3.

### Example 3

### Evaluation and Results of OsMADS18 Under the Control of the Rice OSH1 Promoter

Approximately 15 to 20 independent T0 rice transformants were generated. The primary transformants were transferred from a tissue culture chamber to a greenhouse for growing and harvest of T1 seed. 5 events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. 4 T1 events were further evaluated in the T2 generation following the same evaluation procedure as for the T1 generation but with more individuals per event.

Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and for an overall effect of the gene, also known as a global gene effect. The threshold for significance for a true global gene effect was set at a 5% probability level for the F-test. A significant F-test value points to a gene effect, meaning that it is not only the presence or position of the gene causing the differences in phenotype.

Seed-Related Parameter Measurements

The mature primary panicles were harvested, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) was extrapolated from the number of filled seeds counted and their total weight. Individual seed parameters (including width, length, area, weight) were measured using a custom-made device consisting of two main components, a weighing and imaging device, coupled to software for image analysis.

The tables of results (Tables 5 and 6) below show the p values from the F test for the T1 and T2 generations. The percentage difference between the transgenics and the corresponding nullizygotes is also shown.

The TKW is significantly increased in both the T1 and the T2 generations (Tables 5 and 6). In parallel, an increase in individual seed area is observed, contributing to the observed TKW increase. This increase in seed area is due to a significant increase in individual seed length (Tables 5 and 6), as individual seed width is not significantly changed (data not shown).

TABLE 6
Results of the T1 generation

| | Number of lines | | |
|---|---|---|---|
| | showing an increase | % Difference | P value of F test |
| Thousand kernel | 5 out 5 | 4 | 0.0123 |
| weight | | | |
| Average seed area | 4 out 5 | 3 | 0.0007 |
| Average seed | 3 out 5 | 2 | 0.0001 |
| length | | | |

TABLE 7
Results of the T2 generation

| | Number of lines | | |
|---|---|---|---|
| | showing a | | |
| | positive difference | % Difference | P value |
| Thousand kernel weight | 2 out 4 | 2 | 0.0109 |
| Average seed area | 2 out 4 | 3 | <0.0001 |
| Average seed lenghth | 2 out 4 | 3 | <0.0001 |

1. A method for increasing yield of a plant relative to a corresponding wild type plant, comprising increasing activity in a plant of an OsMADS18-like polypeptide or a homologue thereof and selecting for a plant having increased yield, wherein said OsMADS18-like polypeptide:
(i) is a monocot type II MADS-box transcription factor of the SQUAMOSA lade; and
(ii) has DNA and protein binding activity; and
(iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and
(iv) has at least 65% sequence identity to the OsMADS18 protein of SEQ ID NO: 2.

2. The method according to claim 1, wherein said increased activity is effected by introducing a genetic modification in the locus of a gene encoding an OsMADS18-like polypeptide or a homologue thereof.

3. The method according to claim 2, wherein said genetic modification is effected by one of: site-directed mutagenesis, homologous recombination, directed evolution, TILLING or T-DNA activation.

4. A method for increasing yield of a plant relative to a corresponding wild type plant, comprising introducing and expressing in a plant a nucleic acid or a variant thereof, which nucleic acid encodes an OsMADS18-like polypeptide, which OsMADS18-like polypeptide:
(i) is a monocot type II MADS-box transcription factor of the SQUAMOSA lade; and
(ii) has DNA and protein binding activity; and
(iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and
(iv) has at least 65% sequence identity to the OsMADS18 protein of SEQ ID NO: 2.

5. The method according to claim 4, wherein said variant is a portion of an OsMADS18-like nucleic acid or a sequence capable of hybridising to an OsMADS18-like nucleic acid, which portion or hybridising sequence or complement thereof encodes a monocot type II MADS-box transcription factor of the SQUAMOSA clade having DNA and protein binding activity and comprising:
(i) the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and/or
(ii) having at least 65% sequence identity to the OsMADS18 protein of SEQ ID NO: 2.

6. The method according to claim 4, wherein said OsMADS18-like nucleic acid or variant thereof is overexpressed in a plant.

7. The method according to claim 4, wherein said OsMADS18-like nucleic acid or variant thereof is of plant origin.

8. The method according to claim 4, wherein said variant encodes an orthologue or paralogue of an OsMADS18 protein of SEQ ID NO: 2.

9. The method according to claim 4, wherein said OsMADS18-like nucleic acid or variant thereof is operably linked to an early shoot apical meristem promoter.

10. The method according to claim 9, wherein said early shoot apical meristem promoter is an OSH1 promoter.

11. The method according to claim 1, wherein said increased yield is selected from any one or more of: (i) increased thousand kernel weight (TKW); (ii) increased seed size; (iii) increased seed volume; (iv) increased seed area; (v) increased seed length; and (vi) increased seed biomass.

12. A plant obtained by the method according to claim 1.

13. A construct comprising:
(a) a nucleic acid encoding an OsMADS18-like polypeptide or variant thereof, which OsMADS18-like polypeptide:
(i) is a monocot type II MADS-box transcription factor of the SQUAMOSA clade; and
(ii) has DNA and protein binding activity; and
(iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and
(iv) has at least 65% sequence identity to the OsMADS18 protein of SEQ ID NO: 2; and
(b) one or more control sequences capable of driving expression of the nucleic acid sequence of (a); and optionally
(c) a transcription termination sequence.

14. The construct according to claim 13, wherein said control sequence is an early shoot apical meristem promoter.

15. The construct according to claim 14, wherein said early shoot apical meristem promoter is an OSH1 promoter.

16. The construct according to claim 15, wherein said OSH1 promoter is as represented by SEQ ID NO: 22.

17. (canceled)

18. A plant transformed with the construct claim 13.

19. A method for the production of a transgenic plant having increased yield relative to a corresponding wild type plant, which method comprises:
(a) introducing and expressing in a plant, plant part or plant cell a nucleic acid encoding an OsMADS18-like polypeptide or variant thereof which:
(i) is a monocot type II MADS-box transcription factor of the SQUAMOSA lade; and
(ii) has DNA and protein binding activity; and
(iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and
(iv) has at least 65% sequence identity to the OsMADS18 protein of SEQ ID NO: 2; and
(b) cultivating the plant, plant part or plant cell under conditions promoting plant growth and development.

20. A transgenic plant having increased yield resulting from a nucleic acid or a variant thereof introduced into said plant, which nucleic acid encodes an OsMADS18-like polypeptide that:
(i) is a monocot type II MADS-box transcription factor of the SQUAMOSA clade; and
(ii) has DNA and protein binding activity; and
(iii) comprises the motif LPPWMLRT (SEQ ID NO: 18) in the last 15 amino acids of the C terminus of the protein, allowing for one amino acid substitution anywhere in the motif but not in the second and fourth positions of the motif, which are respectively a proline and a tryptophan; and
(iv) has at least 65% sequence identity to the OsMADS18 protein of SEQ ID NO: 2.

21. The transgenic plant according to claim 20, wherein said plant is a monocotyledonous plant.

22. Harvestable parts of the transgenic plant of claim 21.

23. Harvestable parts according to claim 22, wherein said harvestable parts are seeds which are true breeding for the nucleic acid encoding the OsMADS18-like polypeptide.

24. Products derived from a plant according to claim 21 and/or from harvestable parts of said plant.

25-26. (canceled)

27. A method of selecting a plant with increased yield relative to a corresponding wild type plant, comprising utilizing an OsMADS18-like nucleic acid/gene or variant thereof, or use an OsMADS18-like polypeptide or homologue thereof, as a molecular marker.

28. The method of claim 4, wherein said OsMADS18-like nucleic acid or variant thereof is from a monocotyledonous plant.

29. The method of claim 28, wherein said monocotyledonous plant is from the family Poaceae.

30. The method of claim 28, wherein said monocotyledonous plant is Oryza sativa.

31. The transgenic plant of claim 20, wherein the plant is selected from the group consisting of sugar cane, rice, maize, wheat, barley, millet, rye, oats or sorghum.


SEQUENCE LISTING (44 sequences)

SEQ ID NO: 1 - DNA, 750 bp, Oryza sativa
  misc_feature: OsMADS18 (AF091458)
atggggagagggccggtgcagctgcggcggatcgagaacaagataaacaggcaggtgaccttctccaagcggaggaacgggctgctgaagaaggcgcacgagatctccgtgctctgtgacgccgacgtcgcgctcatcgtcttctccaccaagggcaagctctacgagttctccagccactccagtatggaagggatccttgaacgctaccagcgttactcgtttgatgaaagagccgtactggagccaaatactgaggaccaggaaaactggggtgatgaatatggaattttgaagtccaaactggatgcacttcagaagagccaaaggcaactcttaggtgaacaattggacacactaacaataaaagaactccagcaattggaacatcaactggaatattctctgaagcatataagatcaaaaaagaatcagcttctgtttgaatcaatttctgagcttcagaagaaggaaaagtcacttaaaaaccagaataatgttctgcaaaagctcatggagacagaaaaggagaaaaacaatgctataataaacactaaccgggaggagcaaaatggagcaacaccaagcacatcatcaccaacaccagtgacggctccagatcccatcccgacaacaaataacagtcaaagccaaccaagaggatcaggggagtcagaagctcaaccgtctccggcacaagcaggcaacagcaagcttccgccatggatgctccggacaagtcacacatga

SEQ ID NO: 2 - protein, 249 aa, Oryza sativa
MGRGPVQLRRIENKINRQVTFSKRRNGLLKKAHEISVLCDADVALIVFSTKGKLYEFSSHSSMEGILERYQRYSFDERAVLEPNTEDQENWGDEYGILKSKLDALQKSQRQLLGEQLDTLTIKELQQLEHQLEYSLKHIRSKKNQLLFESISELQKKEKSLKNQNNVLQKLMETEKEKNNAIINTNREEQNGATPSTSSPTPVTAPDPIPTTNNSQSQPRGSGESEAQPSPAQAGNSKLPPWMLRTSHT

SEQ ID NO: 3 - DNA, 756 bp, Zea mays
  misc_feature: ZMM28 (or m28) (AJ430695)
atggggcgggggccggtgcagctgcgccggatcgagaacaagatcaaccgccaggtgaccttctccaagcgccggaacgggctgctgaagaaggcccacgagatctccgtgctctgcgacgcagaggtcgcgctcatcgtcttctccactaaggggaagctctacgagtactctagccattccagcatggaaggcattcttgagcgttaccagcgttactcatttgaagaaagggcagtacttaacccaagtattgaagaccaggcaaattggggagatgaatatgtccggttaaaatccaaacttgatgcacttcagaagagtcaaaggcagctgttaggagaacaattgagttcactgaccataaaagaactccagcaactggagcaacaactggacagttctttgaagcatattaggtcaagaaagaatcagctcatgttcgattcaatttccgcgcttcagaaaaaggagaaagcacttacagatcaaaacggtgtcctgcaaaagttcatggaggcagagaaggagaaaaacaaggctttgatgaacgcgcagctccgggagcagcaaaatggagcatcaacaagctccccatcactttcaccaccaatagttccagattccatgccaactctaaatatagggccatgtcaacatagaggggcagcagaatctgagtctgaaccgtctcctgctcctgcacaagcaaacaggggcaacctgccaccatggatgctccgcactgtcaagtaa

SEQ ID NO: 4 - protein, 251 aa, Zea mays
MGRGPVQLRRIENKINRQVTFSKRRNGLLKKAHEISVLCDAEVALIVFSTKGKLYEYSSHSSMEGILERYQRYSFEERAVLNPSIEDQANWGDEYVRLKSKLDALQKSQRQLLGEQLSSLTIKELQQLEQQLDSSLKHIRSRKNQLMFDSISALQKKEKALTDQNGVLQKFMEAEKEKNKALMNAQLREQQNGASTSSPSLSPPIVPDSMPTLNIGPCQHRGAAESESEPSPAPAQANRGNLPPWMLRTVK

SEQ ID NO: 5 - DNA, 840 bp, Lolium perenne
  misc_feature: MADS3 (AY198328)
atggggcgcgggccggtgcagctgcggcggatcgagaacaagataaaccgccaggtcaccttctccaagcgccggagcgggctgctcaagaaggcgcacgagatctccgtgctctgcgacgctgaggtcgcgctcatcgtattctccaccaagggaaagctctacgagtactccagccaggacagtaacatggatgtcattcttgaacgttaccagcgttactcattcgaagaaagagctatagtggatcaaaatattggaggccaggcaaattggggagatgaatttggaagtttgaaaattaaactcgatgcactccagaagagtcaaaggcaactcttaggtgaacaattggacccactgaccacaaaagaacttcaacaattggaacagcagctagatagttctttgaagcacataaggtcaagaaagaatcagcttctgtttgagtcaatatctgagcttcagaagaaggagaagtcacttaaagatcagaatggcgtcctgcagaagcacctcgtggagacagaaaaggagaaaagtaacgttttatcgaatattcatcaccgagagcagacgaatggagcagcaaatattcatcgccgggagcagatgaatgaaacaacacatattcataaccaggagcagctcaatggagcaacaacaagctcaccgtcacctacaccagtggcggtcctagattccgtggcaactctaaatattgggtcatctcaatctagagaagcagcaggagaggagccagaatctcagccgtccccggcacaagcaaacagcggcaagctaccaccatggatgctccgcaccatcagtaacagatga

SEQ ID NO: 6 - protein, 279 aa, Lolium perenne
MGRGPVQLRRIENKINRQVTFSKRRSGLLKKAHEISVLCDAEVALIVFSTKGKLYEYSSQDSNMDVILERYQRYSFEERAIVDQNIGGQANWGDEFGSLKIKLDALQKSQRQLLGEQLDPLTTKELQQLEQQLDSSLKHIRSRKNQLLFESISELQKKEKSLKDQNGVLQKHLVETEKEKSNVLSNIHHREQTNGAANIHRREQMNETTHIHNQEQLNGATTSSPSPTPVAVLDSVATLNIGSSQSREAAGEEPESQPSPAQANSGKLPPWMLRTISNR

SEQ ID NO: 7 - DNA, 798 bp, Hordeum vulgare
  misc_feature: m3 (AJ249143)
atggggcgcgggccggtgcagctgcggcggatcgagaacaagataaaccggcaggtgaccttctccaagcggcgcagcgggctgcttaagaaggcgcacgagatctccgtgctctgcgacgccgaggtcgcgctcatcgtcttctccaccaagggcaagctatacgagtactccagccaggacagtagcatggatgtgattcttgaacgttaccaacgttactcatttgaagaaagagctgtactggatccaagtactggagaccaggcaaattggggagacgaatatggaagtttgaagataaaactcgatgcactccagaagagtcaaaggcaactcttaggtgaacaattggacccactcaccacaaaagaacttcaacagttggaacaacagcttgatagttctttgaagcacataaggtcaagaaagaatcaacttctctttgagtcaatatctgagcttcaaaagaaggagaaatcattaaaagatcagaatggcgtcctgcagaagcacctcgtagagacagaaaaggagaaaaataacgttttgtcaaatattcatcaccgggagcagttgaatgaagcaacaaatattcatcaccaggagcagctgagtggagcaacaacaagctcaccgtcacctacaccaccgacggcccaagattccatggcacctccaaatattgggccatatcaatctagaggaggaggggatccagaacctcagccgtcaccagcacaagcaaacaacagcaatctgccaccgtggatgctccgcaccatcggcaacagatga

SEQ ID NO: 8 - protein, 264 aa, Hordeum vulgare
MGRGPVQLRRIENKINRQVTFSKRRSGLLKKAHEISVLCDAEVALIVFSTKGKLYEYSSQDSSMDVILERYQRYSFEERAVLDPSTGDQANWGDEYGSLKIKLDALQKSQRQLLGEQLDPLTTKELQQLEQQLDSSLKHIRSRKNQLLFESISELQKKEKSLKDQNGVLQKHLVETEKEKNNVLSNIHHREQLNEATNIHHQEQLSGATTSSPSPTPPTAQDSMAPPNIGPYQSRGGGDPEPQPSPAQANNSNLPPWMLRTIGN

SEQ ID NO: 9 - DNA, 727 bp, Saccharum officinarum
  misc_feature: ZMM28-like partial DNA (EST CA285442)
  misc_feature (620)..(620): n is a, c, g, or t
  misc_feature (662)..(662): n is a, c, g, or t
  misc_feature (689)..(689): n is a, c, g, or t
cgcaccactcgagtcgagagacgagagccacctgatccatccttccccgtcttcctcttcccacacgtccccatcgccattgctgcgccgagagtgagcgggagagggtaggtgtcgaggcggcggagatggggagcgggccggtgcagctgcgccggatcgagaacaagatcaaccgccaggtgaccttctccaagcgccggaacgggctgctgaagaaggcccacgagatctccgtgctctgcgatgcagaggtcgcgctcatcgtcttctccactaaggggaagctctacgagtactctagccattccagcatggaaggcattcttgagcgttaccagcggtactcatttgaagaaagagcagtacttgacccaagtattgaagaccaggcagattggggagatgaatatgtccggttaaaatccaaacttgatgcacttcagaagagtcaaaggcatctgctaggagaacaattggattcactgaccataaaagaactccagcaactggagcaacaattggacagttctttgaagcatattaggtcaagaaagaatcagctcatgttcgattcaatttctgagcttcagaaaaaggagaaagcacttacagatcanaatggtgtcctgcaaaagctcatggaggcagagaaagagaanaacaatgctttaatgaacgcacacctncgggagcagcaaaatggagcatcaacaggctccccata

SEQ ID NO: 10 - DNA, 680 bp, Saccharum officinarum
  misc_feature: ZMM28-like partial DNA (EST CA200888)
ttcttgagcgttaccagcggtactcatttgaagaaagagcagtacttgacccaagtattgaagaccaggcagattggggagatgaatatgtccagttaaaatccaaacttgatgcacttcagaagagtcaaagacatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatgtagcatcaacaagctccccatcactgtcaccaccaatggttccagattccatgccaactctaaatattgggtcatgtcaacctagagggccaggagaatctgagcctgaaccgtctcctgctcctgtacaagcaaacagcggcaacctgccaccatggatgctccgcactgtcagtaacagatgagctcttcccaatgcagttttgcggctgaacacgacagcaacacacctgccaccgacacgccacggtgacccaggcatcaaccgatgcatgatccctctcgatcctgtgatccaa

SEQ ID NO: 11 - DNA, 483 bp, Saccharum officinarum
  misc_feature: ZMM28-like partial DNA (EST CA247266)
cttattttgcagcatggaaggcattcttgagcgttaccagcggtactcatttgaagaaagagcagtacttgacccaagtattgaagaccaggcagattggggagatgaatatgtccagttaaaatccaaacttgatgcacttcagaagagtcaaagacatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatgtagcatcaacaagctccccatcactgtcaccaccaatggttccagattccatgccaactctaaatattgggatgtcaac

SEQ ID NO: 12 - DNA, 714 bp, Saccharum officinarum
  misc_feature: ZMM28-like partial DNA (EST CA282968)
cagtacttgacccaagtattaaagaccaggcagattggggagatgaatatgtccggttaaaatccaaacttgatgcacttcagaagagtcaaaggcatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatgtagcatcaacaagctccccatcactgtcaccaccaatggttccagattccatgccaactctaaatattgggtcatgtcaacctagagggccaggagaatctgagcctgaaccgtctcctgctcctgtacaagcaaacagcggcaacctgccaccatggatgctccgcactgtcagtaacagatgagctcttcccaatgcagttttgcggctgaacacgacagcaacacacctgccaccgacacgccacggtgaaccaggcatcaaccgatgcatgatccctctcgatcctgtgatccaataccgggccgtagtactactaattaaagtccaggccccgctcaaattgctttacatggttgattgaaagttct

SEQ ID NO: 13 - DNA, 650 bp, Saccharum officinarum
  misc_feature: ZMM28-like partial DNA (EST CA299090)
  misc_feature (626)..(626): n is a, c, g, or t
gaagctctacgagtactctagccattccagcatggaaggcattcttgagcgttaccagcggtgctcatttgaagaaagagcagtacttgacccaagtattgaagaccaggcagattggggagatgaatatgtccggttaaaatccaaacttgatgcacttcagaagagtcaaaggcatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatggagcatcaacaagctccccatcactgtcaccaccaatggttccagattccatgccaactctaaatattgggtcatgtcaacctagagggccaggagaatctgagcctgaaccgtctcctgctcctgtacaagcaaacagcgggaacctgccaccatggatactccgcactgtcagtaacagatgagctctttccaatgcagttntgcggctgaactcgacagcaacac

SEQ ID NO: 14 - DNA, 762 bp, Saccharum officinarum
  misc_feature: ZMM28-like reconstituted DNA version 1
atggggagcgggccggtgcagctgcgccggatcgagaacaagatcaaccgccaggtgaccttctccaagcgccggaacgggctgctgaagaaggcccacgagatctccgtgctctgcgatgcagaggtcgcgctcatcgtcttctccactaaggggaagctctacgagtactctagccattccagcatggaaggcattcttgagcgttaccagcggtactcatttgaagaaagagcagtacttgacccaagtattgaagaccaggcagattggggagatgaatatgtccggttaaaatccaaacttgatgcacttcagaagagtcaaaggcatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatggagcatcaacaagctccccatcactgtcaccaccaatggttccagattccatgccaactctaaatattgggtcatgtcaacctagagggccaggagaatctgagcctgaaccgtctcctgctcctgtacaagcaaacagcggcaacctgccaccatggatgctccgcactgtcagtaacagatga

SEQ ID NO: 15 - protein, 253 aa, Saccharum officinarum
  MISC_FEATURE: ZMM28-like reconstituted protein version 1
MGSGPVQLRRIENKINRQVTFSKRRNGLLKKAHEISVLCDAEVALIVFSTKGKLYEYSSHSSMEGILERYQRYSFEERAVLDPSIEDQADWGDEYVRLKSKLDALQKSQRHLLGEQLDSLTIKELQQLEQQLDSSLKHIRSRKNQLMFDSISELQKKEKALTDQNGVLQKLMEAEKEKNNALMNAHLREQQNGASTSSPSLSPPMVPDSMPTLNIGSCQPRGPGESEPEPSPAPVQANSGNLPPWMLRTVSNR

SEQ ID NO: 16 - DNA, 762 bp, Saccharum officinarum
  misc_feature: ZMM28-like reconstituted DNA version 2
atggggagcgggccggtgcagctgcgccggatcgagaacaagatcaaccgccaggtgaccttctccaagcgccggaacgggctgctgaagaaggcccacgagatctccgtgctctgcgatgcagaggtcgcgctcatcgtcttctccactaaggggaagctctacgagtactctagccattccagcatggaaggcattcttgagcgttaccagcggtactcatttgaagaaagagcagtacttgacccaagtattgaagaccaggcagattggggagatgaatatgtccggttaaaatccaaacttgatgcacttcagaagagtcaaaggcatctgttaggagaacaattggattcactgaccataaaagaactccagcaactggagcaacaattggacagttctttgaagcatattaggtcaagaaagaatcagctcatgttcgattcaatttccgagcttcagaaaaaggagaaagcacttacagatcaaaatggtgtcctgcaaaagctcatggaggcagagaaggagaaaaacaatgctttaatgaacgcacacctccgggagcagcaaaatgtagcatcaacaagctccccatcactgtcaccaccaatggttccagattccatgccaactctaaatattgggtcatgtcaacctagagggccaggagaatctgagcctgaaccgtctcctgctcctgtacaagcaaacagcggcaacctgccaccatggatgctccgcactgtcagtaacagatga

SEQ ID NO: 17 - protein, 253 aa, Saccharum officinarum
  MISC_FEATURE: ZMM28-like reconstituted protein version 2
MGSGPVQLRRIENKINRQVTFSKRRNGLLKKAHEISVLCDAEVALIVFSTKGKLYEYSSHSSMEGILERYQRYSFEERAVLDPSIEDQADWGDEYVRLKSKLDALQKSQRHLLGEQLDSLTIKELQQLEQQLDSSLKHIRSRKNQLMFDSISELQKKEKALTDQNGVLQKLMEAEKEKNNALMNAHLREQQNVASTSSPSLSPPMVPDSMPTLNIGSCQPRGPGESEPEPSPAPVQANSGNLPPWMLRTVSNR

SEQ ID NO: 18 - protein, 8 aa, Artificial sequence
  amino acid consensus sequence monocot RT C-terminal motif
LPPWMLRT

SEQ ID NO: 19 - protein, 8 aa, Artificial sequence
  amino acid consensus sequence monocot SH C-terminal motif
LPPWMLSH

SEQ ID NO: 20 - DNA, 50 bp, Artificial sequence
  primer prm05821
ggggacaagtttgtacaaaaaagcaggcttaaacaatggggagagggccg

SEQ ID NO: 21 - DNA, 50 bp, Artificial sequence
  primer prm05822
ggggaccactttgtacaagaaagctgggttgagtggagtgacgtttgaga

SEQ ID NO: 22 - DNA, 1042 bp, Oryza sativa
  misc_feature: OSH1 promoter
ggggagttaggaaccttgacatacaaccaatgataccattttttttcaagcagctagaggtacagaggttcatattttttagatggctcattagtgatattttagtcaaaaatttcagaagtacggccacgggtgatggcctgaactaatattttattcgaggtgccgctacatcatcgtctaaagtacacgcaagattcaacggaaaaaagaaaccgatcgatcgagatcagttagttaatgaaataactagatcaactcatgtcgtcaaaaacaaaagatgctcatctatggacaacacacgctgatgattcgatcatcaaacaaaggtggtagtagtagtaaagcgtatcgtgtttctcatcagaaagaagaattaaagaaaaaactaatcccgtctcgcgagccagagaaaattccctacaaaagccactcctttgatttgacatgcaaaagcaaggctccactcctctactaccctacaactacacaacactgtctctctatctccaaaggcagtagctgtattggcttccagcttttcctctctacctctaatgatagcttggagcaagttcaatagtatagctaactactagctctaattcatctataatcaatctaatagcttattcatacaatagttatatactacattattaatatctggtcccatctatcatacacactgagtctgtgctatagctgactacaaatctgtagcccgctgctcttctctcttcatttatcttcttaaaatatatttgcagctggcttatggcttatagcttgatattgagagggagaggagtgagagctagctcagctcagctcaaggtaaacaacaaggcacactcttcctacctcttctccggttcttcctttttcctctttctcttcgtccaagaacttcacctcaatagctcgagctacggcctaacttttgcgttgcgcaggagagctcgatcgctgcaccaatacttcactggagatcgcctagctgcagctagctagcttatcctgcgtgtcttgagcttctctc

SEQ ID NO: 23 - protein, 256 aa, Arabidopsis thaliana
MGRGRVQLKRIENKINRQVTFSKRRAGLLKKAHEISVLCDAEVALVVFSHKGKLFEYSTDSCMEKILERYERYSYAERQLIAPESDVNTNWSMEYNRLKAKIELLERNQRHYLGEDLQAMSPKELQNLEQQLDTALKHIRTRKNQLMYESINELQKKEKAIQEQNSMLSKQIKEREKILRAQQEQWDQQNQGHNMPPPLPPQQHQIQHPYMLSHQPSPFLNMGGLYQEDDPMAMRRNDLELTLEPVYNCNLGCFAA

SEQ ID NO: 24 - protein, 253 aa, Arabidopsis thaliana
MGRGRVELKRIENKINRQVTFSKRRTGLLKKAQEISVLCDAEVSLIVFSHKGKLFEYSSESCMEKVLERYERYSYAERQLIAPDSHVNTNWSMEYSRLKAKIELLERNQRHYLGEELEPMSLKDLQNLEQQLETALKHIRSRKNQLMNESLNHLQRKEKEIQEENSMLTKQIKERENILRTKQTQCEQLNRSVDDVPQPQPFQHPHLYMIAHQTSPFLNMGGLYQGEDQTAMRRNNLDLTLEPIYNYLGCYAA

SEQ ID NO: 25 - protein, 248 aa, Antirrhinum majus
MGRGKVQLKRIENKINRQVTFSKRRGGLLKKAHELSVLCDAEVALIVFSNKGKLFEYSTDSCMDRILEKYERYSFAERQLVSNEPQSPANWTLEYSKLKARIELLQRNHRHYMGEDLDSMSLKEIQSLEQQLDTALKNIRTRKNQLLYDSISELQHKEKAIQEQNTMLAKKIKEKEKEIAQQPQWEHHRHHTNASIMPPPPQYSMAPQFPCINVGNTYEGEGANEDRRNELDLTLDSLYSCHLGCFAA

SEQ ID NO: 26 - protein, 244 aa, Lycopersicon esculentum
MGRGKVELRKIENKINRQVTFSKRRGGLVKKAHEISVLCDAEVALIVFSQKGKIFEYSSDSCMEQILERYERYSYAERRLLANNSESPVQENWSLEYTKLKARIDLLQRNHKHYMGEDLDSMSLKDLQNLEQQLDSALKLIRSRKNQLMHESISELQKKERAILEENNMLTKKIKEKDKIVEQQGEWHQQTNQVSTSTSFLLQPHQCLNMGGNYQDEVAEARRNNELDLNLDSLYPLYNMNKHL

SEQ ID NO: 27 - protein, 265 aa, Hordeum vulgare
MGRGPVQLRRIENKINRQVTFSKRRSGLLKKAHEISVLCDAEVALIVFSTKGKLYEYSSQDSSMDVILERYQRYSFEERAVLDPSTGDQANWGDEYGSLKIKLDALQKSQRQLLGEQLDPLTTKELQQLEQQLDSSLKHIRSRKNQLLFESISELQKKEKSLKDQNGVLQKHLVETEKEKNNVLSNIHHREQLNEATNIHHQEQLSGATTSSPSPTPPTAQDSMAPPNIGPYQSRGGGDPEPQPSPAQANNSNLPPWMLRTIGNR

SEQ ID NO: 28 - protein, 249 aa, Oryza sativa
MGRGPVQLRRIENKINRQVTFSKRRNGLLKKAHEISVLCDADVALIVFSTKGKLYEFSSHSSMEGILERYQRYSFDERAVLEPNTEDQENWGDEYGILKSKLDALQKSQRQLLGEQLDTLTTKELQQLEHQLEYSLKHIRSKKNQLLFESISELQKKEKSLKNQNNVLQKLMETEKEKNNAIINTNREEQNGATPSTSSPTPVTAPDPIPTTNNSQSQPRGSGESEAQPSPAQAGNSKLPPWMLRTSHT

SEQ ID NO: 29 - protein, 246 aa, Oryza sativa
MGRGKVQLKRIENKINRQVTFSKRRSKLLKKANEISVLCDAEVALIIFSTKGKLYEYATDSCMDKILERYERYSYAEKVLISAESDTQGNWCHEYRKLKAKVETIQKCQKHLMGEDLESLNLKELQQLEQQLENSLKHIRSRKSQLMLESINELQRKEKSLQEENKVLQKELVEKQKVQKQQVQWDQTQPQTSSSSSSFMMREALPTTNISNYPAAAGERIEDVAAGQPQHERIGLPPWMLSHING

SEQ ID NO: 30 - protein, 245 aa, Zea mays
MGRGKVQLKRIENKINRQVTFSKRRSGLLKKAHEISVLCDAEVALIIFSTKGKLYEYSTDSCMDKILDRYERYSYAEKVLISAESETQGNWCHEYRKLKAKVETIQKCQKHLMGEDLETLNLKELQQLEQQLESSLKHIRTRKNQLMLESISELQRKEKSLQEENKVLQKELAEKQKAQRKQVQWGQTQQQTSSSSSCFVIREAAPTTNISIFPVAAGGRLVEGAAAQPQARVGLPPWMLSHLSS

SEQ ID NO: 31 - protein, 245 aa, Zea mays
MGRGKVQLKRIENKINRQVTFSKRRSGLLKKAHEISVLCDAEVALIIFSTKGKLYEYSTDSCMDKILERYERYSYAEKVLISAEYETQGNWCHEYRKLKAKVETIQKCQKHLMGEDLETLNLKELQQLEQQLESSLKHIRTRKSQLMVESISALQRKEKSLQEENKVLQKELAEKQKDQRQQVQRDQTQQQTSSSSTSFMLREAAPTTNVSIFPVAAGGRVVEGAAAQPQARVGLPPWMLSHLSC

SEQ ID NO: 32 - protein, 244 aa, Hordeum vulgare
MGRRKVQLKRIENKINRQVTFSKRRSGLLKKAHEISVLYDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRARKNQLMHESISELQKKERSLQEENKVLQKELVEKQKAQAAQQDQTQPQTSSSSSSFMMRDAPPVADTSNHPAAAGERAEDVAVQPQVPLRTALPLWMVSHING

SEQ ID NO: 33 - protein, 244 aa, Triticum aestivum
MGRGKVQLKRIENKINRQVTFSKRRSGLLKKAHEISVLCDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRSRKNQLMHESISELQKKERSLQEENKVLQKELVEKQKAQAAQQDQTQPQTSSSSSSFMMRDAPPAAATSIHPAAAGERAGDAAVQPQAPPRTGLPLWMVSHING

SEQ ID NO: 34 - protein, 267 aa, Oryza sativa
MGRGKVQLKRIENKINRQVTFSKRRNGLLKKAHEISVLCDAEVAAIVFSPKGKLYEYATDSRMDKILERYERYSYAEKALISAESESEGNWCHEYRKLKAKIETIQKCHKHLMGEDHESLNLKELQQLEQQLESSLKHIISRKSHLMLESISELQKKERSLQEENKALQKELVERQKNVRGQQQVGQWDQTQVQAQAQAQPQAQTSSSSSSMLRDQQALLPPQNICYPPVMMGERNDAAAAAAVAAQGQVQLRIGGLPPWMLSHLNA

SEQ ID NO: 35 - protein, 270 aa, Zea mays
MGRGKVQLKRIENKINRQVTFSKRRNGLLKKAHEISVLCDAEVAVIVFSPKGKLYEYASDSRMDKILERYERYSYAEKALISAESESEGNWCHEYRKLKAKIETIQRCHKHLMGEDLESLNPKELQQLEQQLESSLKHIRSRKSHLMAESISELQKKERSLQEENKILQKELSERQKAVASRQQQQQQVQWDQQTQVQVQTSSSSSSFMMRQDQQGLPPPQNICFPPLSIGERGEEVAAAAQQQLPPPGQAQPQLRIAGLPPWMLSHLNA

SEQ ID NO: 36 - protein, 273 aa, Zea mays
MGRGKVQLKRIENKINRQVTFSKRRNGLLKKAHEISVLCDAEVAVIVFSPKGKLYEYATDSRMDKILERYERYSYAEKALISAESESEGNWCHEYRKLKAKIETIQKCHKHLMGEDLESLNPKELQQLEQQLDSSLKHIRSRKSHLMAESISELQKKERSLQEENKALQKELAERQKAVASRQQQQQQQVQWDQQTHAQAQTSSSSSSFMMRQDQQGLPPPHNICFPPLTMGDRGEELAAAAAAQQQQPLPGQAQPQLRIAGLPPWMLSHLNA

SEQ ID NO: 37 - protein, 247 aa, Oryza sativa
MGRGKVQVRRIENEVSRQVTFSKRRPGLLKKAHEIAVLCDVDVAAIVFSAKGNLFHYASSHTTMERILEKYDRHELLSEGNNVIEEFPELEGSMSYDHIKLRGRIEALKKSQRNLMGQELDSLTLQDIQQLENQIDTSLNNIRSRKNNLLLKSIAELRQKEKLLMEKNTILEKKITELETLHTCIRASPTKAAAPPACNTADAFVPNLNICCGDSGEPETVTAPLGWTSSNNGLPWWMLQSSSNGKS

SEQ ID NO: 38 - protein, 249 aa, Arabidopsis thaliana
MGRGRVQLRRIENKIRRQVTFSKRRTGLVKKAQEISVLCDAEVALIVFSPKGKLFEYSAGSSMERILDRYERSAYAGQDIPTPNLDSQGECSTECSKLLRMIDVLQRSLRHLRGEEVDGLSIRDLQGVEMQLDTALKKTRSRKNQLMVESIAQLQKKEKELKELKKQLTKKAGEREDFQTQNLSHDLASLATPPFESPHELRRTISPPPPPLSSGDTSQRDGVGEVAAGTLIRRTNATLPHWMPQLTGE

SEQ ID NO: 39 - protein, 246 aa, Petunia hybrida
MGRGRVQMKRIENKINRQVTFSKRRSGLLKKAHEISVLCDAEVGLIVFSTKGKLFEYATDSCMERILERYERYSYAERQLVSTDHSSPGSWNLEHAKLKARIEVVQRNQRHYMGEDLDSLSMKDLQNLEQQLDSSLKHIRSRKNQLMHESISELQKKDKSLQEQNNLLSKKVKEREKELAQQTQWEQQNNHHEINSSSSFVLPQPLDSPHLGEAYQSTVDNGEVEGASQQQPANTMPPWMLRHLNG

SEQ ID NO: 40 - protein, 250 aa, Solanum tuberosum
MGRGRVQLKRIENKINRQVTFSKRRSGLLKKAHEISVLCDAEVGLIVFSTKGKLFEYANDSCMERLLERYERYSFAERQLVPTDHTSPGSWTLEHAKLKARLEVLQRNQKHYVGEDLESLNMKELQNLEHQLDSALKHIRSRKNQLMHESISVLQKQDRALQEQNNQLSKKVKEREKEVAQQNQWDQQNHEINSSTFVLPQQLDSPHLGEAYQNTNVVDNGEVEGGNSSQQQGAANNTVMPQWMLRHLNG

SEQ ID NO: 41 - protein, 244 aa, Betula pendula
MGRGRVQLKRIENKINRQVTFSKRRSGLLKKAHEISVLCDAEVALIVFSTKGKLFEYSTDSCMERILERYERYSYADRQLLANDLEQNGSWTLEHAKLKARIEVLQRNQKHFVGEDLDSLSLKELQNLEQQLDSALKHIRSRKNQLMYESISELQRKDKALQEQNNVLAKKVKEKEKELAQQAQWEQQSHTLDSVPSLLPQPLQSSLNIGGSQQARGNGRVDEGTPPHRANALLPPWMLRHLNQ

SEQ ID NO: 42 - protein, 252 aa, Antirrhinum majus
MGRGRVQLKRIENKISRQVTFSKRRSGLLKKAHEISVLCDAEVALIVFSTKGKLFEYSTESSMERILERYERYSYAEKKLTSDSHEPEENWCLEYPKLVARIELLERNIRNYVGEDLDHLSMRELQSLEQQLDTALKRTRTRKNQLMHESISQLQKKERSLQDQNNILAKKIKDNEKQQNEKQQDVHEGFAQSSSSINMLLQPPATQLHAVPCLPISGGFQQTVRVEEGGDRTRIADSRSHIPPWLLQHVNQ

SEQ ID NO: 43 - protein, 241 aa, Sinapsis alba
MGRGRVQLKRIENKINRQVTFSKRRSGLLKKAHEISVLCDAEVALVIFSSKGKLFEYSTDSCMEKILERYDRYLYSDKQLVGRDISQSENWVLEHAKLKARVEVLEKNKRNFMGEDLDSLSLKELQSLEHQLHAAIKSIRSRKNQAMFESISALQKKDKVLQDHNNALLKKIKEREKNTVHQEVQLIQCSNNSSILQPQYCLTSSRDGFVGRVEGENEGASSLAEPNSLLPAWMLRPTTNE

SEQ ID NO: 44 - protein, 242 aa, Arabidopsis thaliana
MGRGRVQLKRIENKINRQVTFSKRRSGLLKKAHEISVLCDAEVALIVFSSKGKLFEYSTDSCMERILERYDRYLYSDKQLVGRDVSQSENWVLEHAKLKARVEVLEKNKRNFMGEDLDSLSLKELQSLEHQLDAAIKSIRSRKNQAMFESISALQKKDKALQDHNNSLLKKIKEREKKTGQQEGQLVQCSNSSSVLLPQYCVTSSRDGFVERVGGENGGASSLTEPNSLLPAWMLRPTTTNE